(19) United States
(12) Patent Application Publication
(10) Pub. No.: US 2002/0155453 A1
(43) Pub. Date: Oct. 24, 2002

(54) Method for generating diversity

(76) Inventors: Julian Edward Sale, Cambridge (GB); Michael Samuel Neuberger, Cambridge (GB); Sarah Jane Cumbers, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 09/879,813

(22) Filed: Jun. 11, 2001

Related U.S. Application Data
(63) Continuation-in-part of application No. 09/828,717, filed on Apr. 6, 2001.
Application No. 09/828,717 is a continuation-in-part of application No. PCT/GB99/03358, filed on Oct. 8, 1999.

(30) Foreign Application Priority Data
Oct. 9, 1998 (GB) 9822104.7
Jan. 12, 1999 (GB) 9901141.3
Jun. 9, 1999 (GB) 9913435.5

(51) Int. Cl. C12Q 1/68; C12N 15/87

(52) U.S. Cl. 435/6; 435/455

(57) ABSTRACT

The invention relates to a method for preparing an antibody-producing cell line capable of directed constitutive hypermutation of a specific nucleic acid region, comprising the steps of: a) screening a clonal cell population for V gene diversity; b)isolating one or more cells which display V gene diversity and comparing the rate of accumulation of mutations in the V genes and other genes of the selected cells; and c) selecting a cell in which the rate of V gene mutation exceeds that of other gene mutations.

### FIELD OF THE INVENTION

The present invention relates to a method for generating diversity in a gene or gene product by exploiting the natural somatic hypermutation capability of antibody-producing cells, as well as to cell lines capable of generating diversity in defined gene products.

### BACKGROUND OF THE INVENTION

Many in vitro approaches to the generation of diversity in gene products rely on the generation of a very large number of mutants which are then selected using powerful selection technologies. For example, phage display technology has been highly successful as providing a vehicle that allows for the selection of a displayed protein (Smith, 1985, Science 228: 1315-7; Bass et al., 1990, Proteins. 8: 309-314; McCafferty et al., 1990, Nature 348: 552-4; for review see Clackson and Wells, 1994, Trends Biotechnol. 12: 173-84). Similarly, specific peptide ligands have been selected for binding to receptors by affinity selection using large libraries of peptides linked to the C terminus of the lac repressor Lacl (Cull et al., 1992, Proc. Natl. Acad. Sci. U.S.A, 89: 1865-9). When expressed in E. coli the repressor protein physically links the ligand to the encoding plasmid by binding to a lac operator sequence on the plasmid. Moreover, an entirely in vitro polysome display system has also been reported (Mattheakis et al., 1994, Proc. Natl. Acad. Sci. USA 91: 9022-6) in which nascent peptides are physically attached via the ribosome to the RNA which encodes them.

In vivo, the primary repertoire of antibody specificities is created by a process of DNA rearrangement involving the joining of immunoglobulin V, D and J gene segments. Following antigen encounter in mouse and man, the rearranged V genes in those B cells that have been triggered by the antigen are subjected to a second wave of diversification, this time by somatic hypernutation. This hypennutation generates the secondary repertoire from which good binding specificities can be selected thereby allowing affinity maturation of the humoral immune response.

Artificial selection systems to date rely heavily on initial mutation and selection, similar in concept to the initial phase of V-D-J rearrangement which occurs in natural antibody production, in that it results in the generation of a “fixed” repertoire of gene product mutants from which gene products having the desired activity can be selected.

In vitro RNA selection and evolution (Ellington and Szostak, 1990, Nature 346: 81822), sometimes referred to as SELEX (systematic evolution of ligands by exponential enrichment) (Tuerk and Gold, 1990, Science 249: 505-10) allows for selection for both binding and chemical activity, but only for nucleic acids. When selection is for binding, a pool of nucleic acids is incubated with immobilized substrate. Non-binders are washed away, then the binders are released, amplified and the whole process is repeated in iterative steps to enrich for better binding sequences. This method can also be adapted to allow isolation of catalytic RNA and DNA (Green and Szostak, 1992, Science 258: 1910-1915, for reviews, see Chapman and Szostak, 1994, Curr. Op. Struct. Biol. 4: 618-622; Joyce, 1994, Curr. Op. Structural Biol., 4: 331-336; Gold et al., 1995, Annu. Rev. Biochem. 64: 763-97; Moore, 1995, Nature 374: 766-7). SELEX, thus, permits cyclical steps of improvement of the desired activity, but is limited in its scope to the preparation of nucleic acids.

Unlike in the natural immune system, however, artificial selection systems are poorly suited to any facile form of “affinity maturation”, or cyclical steps of repertoire generation and development. One of the reasons for this is that it is difficult to target mutations to regions of the molecule where they are required, so subsequent cycles of mutation and selection do not lead to the isolation of molecules with improved activity with sufficient efficiency.

Much of what is known about the somatic hypermutation process which occurs during affinity maturation in natural antibody production has been derived from an analysis of the mutations that have occurred during hypermutation in vivo (for reviews, see Neuberger and Milstein, 1995, Curr. Op. Immunol. 7: 248-254.; Weill and Reynaud, 1996, Immunol. Today 17: 92-97; Parham, P. (ed)., 1998, In Immunological Reviews, Vol. 162, Copenhagen, Denmark: Munksgaard). Most of these mutations are single nucleotide substitutions which are introduced in a stepwise manner. They are scattered over the rearranged V domain, though with characteristic hotspots, and the substitutions exhibit a bias for base transitions. The mutations largely accumulate during B cell expansion in germinal centers (rather than during other stages of B cell differentiation and proliferation) with the rate of incorporation of nucleotide substitutions into the V gene during the hypermutation phase estimated at between 10⁻⁴ and 10⁻³ bp⁻¹ generation⁻¹ (McKean et al., 1984, Proc. Natl. Acad. Sci. USA 81: 3180-3184; Berek and Milstein, 1988, Immunol. Rev. 105: 5-26).

The possibility that lymphoid cell lines could provide a tractable system for investigating hypermutation was considered many years ago (Coffino and Scharff, 1971, Proc. Natl. Acad. Sci. USA 68: 219-223; Adetugbo et al., 1977, Nature 265: 299-304; Brüggemann et al., 1982, EMBO J. 1: 629-634). Clearly, it is important that the rate of V gene mutation in the cell-line under study is sufficiently high not only to provide a workable assay but also to be confident that mutations are truly generated by the localized antibody hypermutation mechanism rather than reflecting a generally increased mutation rate as is characteristically associated with many tumors. Extensive studies on mutation have been performed monitoring the reversion of stop codons in V_{H} in mouse pre-B and plasmacytoma cell lines (Wabl et al., 1985, Proc. Natl. Acad. Sci. USA 82: 479-482.; Chui et al., 1995, J. Mol. Biol. 249: 555-563; Zhu et al., 1995, Proc. Natl. Acad. Sci. USA 92: 2810-2814; reviewed by Green et al., 1998, Immunol. Rev. 162: 77-87). The alternative strategy of direct sequencing of the expressed V gene has indicated that V_{H} gene diversification in several follicular, Burkitt and Hodgkin lymphomas can continue following the initial transformation event (Bahler and Levy, 1992, Proc. Natl. Acad. Sci. USA 89: 6770-6774.; Jain et al., 1994, J. Immunol. 153: 45-52; Chapman et al., J omput. Aided Mol. Des. 1996, 10(6): 501-12; Chapman et al., 1995, Blood 85: 2176-2181; Braeuninger et al., 1997, Proc. Natl. Acad. Sci. USA 94: 9337-9342). Direct sequencing has also revealed a low prevalence of mutations in a cloned follicular lymphoma line arguing that V_{H} diversification can continue in vitro (Wu et al., 1995, Scand. J. Immunol. 42: 52-59). None of the reports of constitutive mutation in cell lines cited above provides evidence that the mutations seen are the result of directed hypermutation, as observed in natural antibody diversification, which is concentrated in the V genes, as opposed to a general susceptibility to mutation as described in many tumor cell lines from different lineages.

Recently, hypermutation has been induced in a cell line by Denepoux el al. (1997, Immunity 6: 35-460) by culturing cells in the presence of anti-immunoglobulin antibody and activated T-cells. However, the hypermutation observed was stated to be induced, not constitutive.

### SUMMARY OF THE INVENTION

In one aspect, the invention provides a method for obtaining a cell which directs constitutive hypermutation of a target nucleic acid sequence within the cell. The method comprises screening a cell population for ongoing target sequence diversification and selecting a cell in the cell population in which the rate of mutation of the target sequence exceeds the rate of mutations in non-target sequences by a factor of 100 or more. In one aspect, the cell is a lymphoid cell. Preferably, the cell is derived from a cell which hypermutates in vivo, such as an iimmunoglobulin-expressing cell. Still more preferably, the cell is from a cell line (e.g., such as a Burkitt lymphoma cell line, a follicular lymphoma cell line, or a diffuse large cell lymphoma cell line).

In one aspect, mutation rates are determined by sequencing target genes in cells from the cell population. In another aspect, the target nucleic acid sequence encodes a gene product and hypermutating cells are screened for by selecting for a change in the expression of the gene product in one or more cells in the cell population. For example, hypermutating cells can be identified by selecting for the loss of expression of a gene product which is normally expressed on the surface of the cells. Loss of expression can be detected by contacting the cells with an antibody which specifically binds to the gene product to identify one or more cells which do not bind to the antibody, and which are therefore candidate constitutively hypermutating cells. In one aspect, the target sequence is an immunoglobulin V-gene sequence and the gene product is an immunoglobulin.

In one aspect, the population of cells is exposed to a mutagen. In another aspect, the population of cells expresses a sequence-modifying gene product. For example, the cells can comprise one or more mutated sequences, such as mutated DNA repair genes, which provide the cells with a higher rate of mutation than cells without the mutated sequences. Preferably, the rate of mutation is at least at least two-fold higher, or at least ten-fold higher, than cells without the one or more mutated sequences. Still more preferably, the cells comprising the one or more mutated sequences express at least 10% less of one or more DNA repair proteins than cells without the one or more mutated sequences.

In a preferred aspect, the cells comprise mutations in one or more DNA repair genes selected from the group consisting of Rad51, Rad 51 analogues, Rad51 paralogues, and combinations thereof. In one aspect, the DNA repair genes are selected from the group consisting of Rad51b, Rad51c, and analogues, paralogues, and combinations thereof.

The invention also provides a method for preparing a mutated form of a gene product. The method comprises expressing a nucleic acid encoding the gene product which is operably linked to a hypermutation control sequence in a population of constitutively hypermutating cells in which the rate of mutation of nucleic acids linked to the control sequence exceeds the rate of mutations in sequences not linked to the control sequence by a factor of 100 or more and identifying a cell within the population of cells which expresses a mutated form of the gene product.

In one aspect, one or more clonal populations of cells is generated from an identified cell and a cell is selected from the clonal population which expresses the mutated form of the gene product.

In one aspect, the identified cell or cells constitutively hypermutate an endogenous V gene locus.

In one aspect, the mutated form of the gene product binds to a biomolecule to which the non-mutated form of the gene product does not bind. In another aspect, the mutated form of the gene product is unable to bind to a biomolecule under conditions in which the non-mutated form of the gene product binds to the biomolecule. In still another aspect, the mutated form of the gene product comprises an at least two-fold greater ability to bind to a biomolecule to which the non-mutated form of the gene product binds. In a further aspect, the mutated form of the gene product comprises an at least two-fold lower ability to bind to a biomolecule to which the non-mutated form of the gene product binds.

In another aspect, the gene product is an enzyme and performs a catalytic activity in the presence of a substrate (e.g., converts the substrate to a product). In one aspect, the catalytic activity of the mutated gene product is increased at least two-fold compared to the catalytic activity of the non-mutated gene product. In another aspect, the catalytic activity of the mutated gene product is decreased at least two-fold compared to the catalytic activity of the non-mutated gene product.

In a preferred aspect, the hypermutation control sequence comprises a sequence occurring 3′ of a J gene cluster and comprises at least the Jκ-Cκ intron sequence including the Ei/MAR enhancer element sequence, Cκ, and the E3′ enhancer element. In one aspect, the sequence 3′ of Cκ and 5′ of E3′ further comprises a 7.34 kb deletion.

In one aspect, the nucleic acid encoding the gene product is encoded by an exogenous sequence (e.g., such as a heterologous sequence) operably linked to an endogenous control sequence. In another aspect, the target sequence is an exogenous gene operably linked to the Jκ intron. In a further aspect, the exogenous sequence replaces an endogenous V region coding sequence.

In one aspect, the gene product being mutated is an immunoglobulin. In another aspect, the gene product being mutated is a DNA binding protein.

The invention also provides a cell for directing constitutive hypermutation of a target sequence wherein the cell is a genetically manipulated chicken bursal lymphoma cell in which the rate of nucleic acid mutation at the target sequence exceeds the rate of nucleic acid mutations at non-target sequences by a factor of 100 or more. Preferably, the cell is generated from a DT40 cell. Still more preferably, the cell is selected from the group consisting of Δ xrcc2 DT40 and Δ xrcc3 DT40.

### BRIEF DESCRIPTION OF THE FIGURES

The objects and features of the invention can be better understood with reference to the following detailed description and accompanying drawings.

FIGS. 1A-D show V_{H} diversity in Burkitt lines. FIG. 1A shows sequence diversity in the rearranged V_{H} genes of four sporadic Burkitt lymphoma lines, shown as pie charts. The number of M13 clones sequenced for each cell line is denoted in the center of the pie; the sizes of the various segments depict the proportion of sequences that are distinguished by 0, 1, 2 etc. mutations (as indicated) from the consensus. FIG. 1B shows the presumed dynastic relationship of V_{H} mutations identified in the initial Ramos culture. Each circle (with shading proportional to extent of mutation) represents a distinct sequence with the number of mutations accumulated indicated within the circle. FIG. 1C shows mutation prevalence in the rearranged V_{λ} genes. Two V_{λ} rearrangements are identified in Ramos. Diversity and assignment of germline origin is presented as in FIG. 1A. FIG. 1D shows a comparison of mutation prevalence in the V_{H} and Cμ regions of the initial Ramos culture. Pie charts are presented as in FIG. 1A.

FIGS. 2A-B show constitutive V_{H} diversification in Ramos. FIG. 2A shows diversification assessed by a MutS assay. The mutation prevalence in each population as deduced by direct cloning and sequencing is indicated. FIG. 2B shows the dynastic relationships deduced from the progeny of three independent Ramos clones.

FIG. 3 shows the distribution of unselected nucleotide substitutions along the Ramos V_{H}.

FIGS. 4A-D illustrates that hypermutation in Ramos generates diverse revertible IgM-loss variants. FIG. 4A presents a scheme showing the isolation of IgM-loss variants. FIG. 4B illustrates that multiple nonsense mutations can contribute to V_{H} inactivation. Each V_{H} codon position at which stops are observed in these two populations is listed. FIG. 4C shows the reversion rates of IgM-loss variants. FIG. 4D shows the sequence surrounding the stop codons in the IgM-loss derivatives.

FIGS. 5A-B show IgM-loss variants in Ramos transfectants expressing TdT. FIG. 5A shows western blot analysis of expression of TdT in three pSV-pβBG/TdT and three control transfectants of Ramos. FIG. 5B shows pie charts depicting independent mutational events giving rise to IgM-loss variants.

FIG. 6 is a sequence table summarizing mutations in V_{H} other than single nucleotide substitutions.

FIG. 7 provides a comparison of sequences isolated from V_{H} genes of Ramos cells which have lost anti-idiotype (anti-Id1) binding specificity. Nucleotide substitutions which differ from the starting population consensus are shown in bold. Predicted amino acid changes are indicated, also in bold type.

FIG. 8 is a bar graph showing enrichment of Ramos cells for production of an immunoglobulin with a novel binding specificity, by iterative selection over five rounds.

FIG. 9 is a bar graph showing improved recovery of Ramos cells binding a novel specificity (streptavidin) by increasing the bead:cell ratio.

FIG. 10 is a chart showing increase in recovery of novel binding specificity Ramos cells according to increasing target antigen concentration.

FIG. 11 shows a V_{H} sequence derived from streptavidin-binding Ramos cells. Nucleotide changes observed in comparison with the V_{H} sequence of the starting population, and predicted amino acid changes, are shown in bold.

FIG. 12 shows the amount of IgM in supernatants of cells selected in rounds 4, 6 and 7 of a selection process for streptavidin binding compared to control medium and unselected Ramos cell supernatant.

FIG. 13 shows streptavidin binding of IgM from the supernatants of FIG. 12.

FIG. 14 shows streptavidin binding of supernatants from round 4 and round 6 of a selection for streptavidin binding, analyzed by surface plasmon resonance.

FIG. 15 shows FACS analysis of binding to streptavidin-FITC of cells selected in rounds 4 and 6.

FIG. 16 shows V_{H} and V_{L} sequences of round 6 selected IgM.

FIG. 17 shows FACS analysis of affinity matured Ramos cells selected against streptavidin.

FIG. 18 shows ELISA analysis of affinity-matured Ramos cells.

FIGS. 19A and B show sIgM-loss variants in wild-type and repair deficient DT40. FIG. 19A shows flow cytometric analysis of sIgM heterogeneity in wild-type and repair deficient cells. FIG. 19B shows fluctuation analysis of the frequency of generation of sIgM-loss variants.

FIG. 20 shows an analysis of V_{λ} sequences cloned from sIgM variants of DT40.

FIG. 21 provides an analysis of Ig sequences of unsorted DT40 populations after one month of clonal expansion.

FIG. 22 provides an analysis of sIgM loss variants of DT40 cells deficient in DNA-PK, Ku/70 and Rad51B.

FIG. 23 provides an analysis of naturally-occurring constitutively hypermutating BL cell lines.

### DETAILED DESCRIPTION

The invention provides a method for preparing a cell line for directed constitutive hypermutation of a target nucleic acid sequence, comprising screening a cell population for ongoing target sequence diversification and selecting a cell in which the rate of target nucleic acid mutation exceeds that of other nucleic acid mutations by a factor of 100 or more. The invention also provides a cell line obtained by the method and a method of using the cell line to screen for mutated gene products with a desired activity.

Definitions

The following definitions are provided for specific terms which are used in the following written description.

As used herein, “directed constitutive hypermutation” refers to the ability, observed for the first time in experiments reported herein, of certain cell lines to cause alteration of the nucleic acid sequence of one or more specific sections of endogenous or transgene DNA in a constitutive manner, that is without the requirement for external stimulation. In cells capable of directed constitutive hypermutation, sequences outside of the specific sections of endogenous or transgene DNA are not subjected to mutation rates above background mutation rates.

A “target nucleic acid sequence” is a nucleic acid sequence in the cell which is subjected to directed constitutive hypermutation. The target nucleic acid can comprise one or more transcription units encoding gene products, which can be homologous or heterologous to the cell. Exemplary target nucleic acid sequences are immunoglobulin V genes as found in immunoglobulin-producing cells These genes are under the influence of hypermutation-recruiting elements, as described further below, which direct the hypermutation to the target sequence such that sequences operably linked to the elements mutate at a higher rate (at least 100-fold higher) than non-target sequences (i.e., sequences not operably linked to the elements). Preferably, a target sequence is at least 10 base pairs, at least 20 base pairs, at least 100 base pairs, at least 200 base pairs, at least 300 base pairs, or at least 500 base pairs.

As used herein, “a hypermutation-recruiting element” is a sequence which, when operably linked to a target sequence or an endogenous gene sequence, directs one or more mutating factors to the target sequence or endogenous sequence to selectively hypernutate the sequence.

“Hypermutation” refers to the mutation of a nucleic acid in a cell at a rate above background. Preferably, hypermutation refers to a rate of mutation of between 10⁻⁵ and 10⁻³ bp⁻¹ generation⁻¹. This is greatly in excess of background mutation rates, which are of the order of 10⁻⁹ to 10⁻¹⁰ mutations bp⁻¹ generation⁻¹ (Drake et al., 1988, Genetics 148:1667-1686) and of spontaneous mutations observed in PCR. 30 cycles of amplification with Pfu polymerase would produce <0.05×10⁻³ mutations bp⁻¹ in the product, which in the present case would account for less than 1 in 100 of the observed mutations (Lundberg et al., 1991, Gene 108: 1-6).

As used herein, “a control sequence which directs hypermutation” of a target sequence or a “hypermutation control sequence” is a sequence which comprises one or more hypermutating elements and which when operably linked to the target sequence selectively hypermutates the target sequence (e.g., a target gene) and does not hypermutate non-target sequences (e.g., a non-target gene). As used herein, a “control sequence operably linked” to a target sequence refers to a control sequence which is in suitable proximity and orientation relative to a target gene to direct one or more hypermutation factors to the target sequence to constitutively and selectively hypermutate the target sequence.

As used herein, “screening for ongoing target sequence diversification” refers to the determination of the presence of hypermutation in the target nucleic acid region of the cell lines being tested. This can be performed in a variety of ways, including by direct sequencing or by using indirect methods such as the MutS assay (Jolly et al., 1997, NAR 25: 1913-1919) described further below or by monitoring the loss of a gene product encoded by a target sequence being hypermutated (e.g., if the target sequence is an immunoglobulin, by selecting for immunoglobulin loss variants). Cells selected according to this procedure are said to be cells which “display target sequence diversification”. Diversification is said to be “ongoing” where cells identified as displaying sequence diversification continue to diversify their target sequences during additional rounds of cell division.

A “clonal cell population” is a population of cells derived from a single clone, such that the cells would be identical save for mutations occurring therein. Use of a clonal cell population preferably excludes co-culturing with other cell types, such as activated T-cells, with the aim of inducing V gene hypermutation.

As used herein, a “cell derived from” or a cell line derived from” or a “cell generated from” refers to a cell which is the progeny (e.g., first generation, second generation, up to an infinite number of cell generations) of a reference cell and which can comprise one or more genetic alterations when compared to the reference cell.

As used herein, a “cell from a cell line” refers to a continuously proliferating cell or a cell which proliferates for at least 10, at least 20, or at least 30 generations.

As used herein “heterologous” nucleic acids refer to nucleic acids not naturally located in a cell or in a chromosomal site of a cell.

As used herein, a “transgene” is a nucleic acid molecule which is inserted into a cell, such as by transfection or transduction. For example, a “transgene” can comprise a heterologous transcription unit which can be inserted into the genome of a cell at a desired location.

As used herein, an “analogue” refers to a gene which comprises substantial sequence identity to a reference gene (e.g., such as a DNA repair gene) but which still shares the biological activity of the reference gene. For example, an analogue of a DNA repair gene with a nuclease activity will encode a product comprising the same nuclease activities as the founder DNA repair gene product (e.g., such as the ability to function as a 5′-3′ exonuclease), although this activity can differ in degree from the activity of the founder DNA gene product. As used herein, a “paralogue” more specifically refers to a gene which shares not only substantial sequence identity, but also an evolutionary relationship with a reference gene; e.g., a paralogue arises from duplication of a reference gene and can be on the same or a different chromosome as the reference gene.

As used herein, “Rad51 paralogues” and “Rad51 analogues” share at least 50% identity with residues 33-240 of the E. coli RecA protein after maximally aligning the sequences of these proteins using algorithms well known in the art. Preferably, Rad51 analogues and paralogues polymerize on single-stranded DNA to form a right-handed helical nucleoprotein filament which extends DNA by 1.5 times (see, e.g., Benson, et al., 1994, EMBO J. 13: 5764-5771). Rad51 paralogues and analogues promote homologous pairing and strand exchange in an ATP dependent reaction.

As used herein, a “sequence-modifying gene product” is a gene product whose expression enhances the mutation rate in a cell by at least two fold compared to a cell which does not express the gene product.

As used herein, “genetically engineered” or “genetically manipulated” refers to a change in a sequence which has been introduced in vitro; e.g., by cloning, by in vitro recombination systems, and the like. A “change” can be a mutation in the sequence (i.e., a substitution, deletion, insertion, rearrangement) or can be the association of a sequence with other sequences with which the sequence is not normally associated (e.g., such as vector sequences, different promoter sequences, enhancer elements, intron sequences, termination sequences, and the like). A genetically engineered or genetically manipulated nucleic acid sequence can be introduced into a cell and can be maintained extrachromosomally or can be integrated into the genome of the cell. A “genetically engineered “or “manipulated” nucleic acid sequence can be one which is not naturally found in the cell or can be a sequence which is naturally found in the cell, but which is altered in vitro, and re-integrated into the cell's genome by homologous or non-homologous recombination. When the sequence is re-introduced into the cell and re-integrates into the genome by homologous recombination, the sequence can result in the alteration (e.g., deletions, rearrangements, insertions, substitutions) of sequences at the insertion site, i.e., resulting in alteration of the endogenous sequence. When this occurs, the endogenous sequence also can be said to be “genetically engineered” or “manipulated”. A “disrupted” sequence is a sequence which no longer produces a functional gene product.

As used herein, a “mutated form of a gene product” refers to the gene product encoded by a hypermutated gene.

As used herein, a mutated form of a gene product with a “desired activity” refers to a mutated gene product having an activity which is significantly different from the activity of the non-mutated gene product. A desired activity may be different in kind, i.e., an activity which the non-mutated gene product did not have or the loss of an activity which the non-mutated gene product did have. A desired activity also can be different in amount from an activity which was possessed by a non-mutated gene product. For example, a mutated form of a gene product can have at least two-fold, four-fold, five-fold, 10-fold, 20-fold, 30-fold more, 50-fold, and 100-fold more or less activity than a non-mutated gene product, or at least 10%, 20%, 30%, 40%, 50%, 60%, 70%, 80%, 90%, or 100% more or less activity than a non-mutated gene product. Generally, a desired activity which is different in amount can be any difference in activity from the activity of the non-mutated gene product which is statistically significant as determined using standard statistical methods, such as a student's t test and/or ANOVA, defining statistically significant differences where p<0.5.

As used herein, “a biomolecule” is a molecule found within a cell, e.g., such as a nucleic acid, peptide, polypeptide, protein, glycoprotein, lipid, steroid, and the like.

Generation of Constitutively Hypermutating Cell Lines

The present invention makes available for the first time a cell line which constitutively hypermnutates selected target nucleic acid regions. This permits the design of systems which produce mutated gene products by a technique which mirrors affinity maturation in natural antibody production. The Ramos Burkitt line described herein constitutively diversifies its rearranged immunoglobulin V gene during in vitro culture. This hypermutation does not require stimulation by activated T cells, exogenously-added cytokines or even maintenance of the B cell antigen receptor.

The rate of mutation (which lies in the range 0.2-1×10⁻⁴ bp⁻¹ generation⁻¹) in this cell line is sufficiently high to readily allow the accumulation of a large database of sequences representing unselected mutations and so reveals that hypermutation in Ramos exhibits most of the features classically associated with immunoglobulin V gene hypermutation in vivo (preferential targeting of mutation to the V; stepwise accumulation of single nucleotide substitutions; transition mutation bias; characteristic mutational hotspots). The large majority of mutations in this unselected database are single nucleotide substitutions, although deletions and duplications (sometimes with a flanking nucleotide substitution) also are detectable. Such deletions and duplications also have been proposed to be generated as a consequence of hypermutation in vivo (Wilson et al., 1998, J. Exp. Med. 187: 59-70; Goosens et al., 1998, Proc. Natl. Acad. Sci. USA 95: 2463-2468; Wu & Kaartinen, 1995, Eur. J. Immunol. 25: 3263-3269).

In a preferred aspect, cells are screened for which constitutively hypermutate a selected nucleic acid region by monitoring mutations in a target sequence within the region. In one aspect, the target sequence comprises an endogenous gene, such as a V gene. Preferably, the cells being screened are derived from antibody-producing cells such as B cells.

Selection of Hypermutating Cells

Hypermutating cells can be selected from a population of cells by a variety of techniques. In one aspect, hypermutating cells are identified by obtaining nucleic acids from a selected cell or a clone of a selected cell and sequencing the target sequence using methods routine in the art. Preferably, nucleic acids are amplified prior to sequencing.

In another aspect, instead of, or in addition to being sequenced, a target nucleic acid is rendered single-stranded, hybridized with a non-mutated target sequence, and contacted with one or more proteins for detecting mismatched sequences (e.g., such as MutS or a resolvase, such as T4 endonuclease). The binding of the one or more proteins at a mismatched site can be detected and used to identify and/or quantitate mismatches in a candidate hypermutated sequence. Alternatively, or additionally, bound nucleic acids can be contacted with an agent which detectably modifies the site of a mismatch and detection of the modification can be used to identify and/or quantitate the presence of a mismatch.

Preferably, hypermutations in a target sequence are detected using a MutS-based assay system. The E. coli MutS protein (GenBank Accession No. U69873) binds to several different types of mismatches (Jiricny et al., 1988, Nucleic Acids Res. 16: 7843-7853; Lishanski et al., 1994, Proc. Natl. Acad. Sci. USA 91:2674-2678; and Jolly, et al., supra) and can be purified from an overproducing strain of E. coli (Su and Modrich, 1986, Proc. Natl. Acad. Sci. USA 83: 5057-5061). In one aspect, amplified target sequences from a candidate constitutively hypermutating cell or clone of such a cell are labeled using biotin (e.g., using biotinylated primers during the amplification process), purified, denatured, and then renatured in the presence of non-mutated sequences. A solid support, such as a sheet of nitrocellulose, nylon membrane, or filter, is incubated with MutS protein (e.g., by spotting MutS protein on the support, using a slot blot or dot blot apparatus) and candidate mutated sequences hybridized to non-mutated sequences are added to the MutS containing portions of the support. The support is then incubated with a streptavidin-bound reporter enzyme (such as horseradish peroxidase) and the activity of the reporter enzyme detected as a means to detect and/or quantitate mutations in the target sequence (see, e.g., as described in Jolly et al., supra).

In another aspect, a candidate constitutively hypermutating cell is identified by screening for the loss of a gene product encoded by the target sequence, since one of the features of hypermutation of target nucleic acids is that the process results in the introduction of stop codons into the target sequence with far greater frequency than would be observed in the absence of hypermutation. In one aspect, loss of the gene product is screened for by immunofluorescence or FACS analysis to detect which cells do not bind an antibody specific for the gene product. Solid phase assays also can be used. In such assays, binding partners which specifically bind to the gene product encoded by the target nucleic acid are bound to a solid support and are used to remove cells which express the gene product, leaving non-expressing cells behind, i.e., candidate constitutively hypermutating cells. Preferably, the binding partners used are antibodies. The solid phase can be any routinely used in the art, such as beads, particles, chips, capillaries, filters and the like, and also can be magnetic or paramagnetic, to facilitate separating desired populations of cells from undesired populations of cells.

In a further aspect, hypermutation in a cell is assayed for by detecting a change in the activity of a gene product encoded by a target gene. For example, in one aspect, the gene product comprises a binding activity and the loss of binding activity of the gene product is screened for. In another aspect, changes in the amount of binding are monitored (e.g., by quantitating the amount of a binding partner bound by the gene product) and used to screen for hypermutating cells. In a further aspect, the gene product is an enzyme and the changes in the catalytic activity of the enzyme is monitored (e.g., as determined by measuring the amount of a substrate converted to product) as a means of identifying hypermutating cells.

In a preferred embodiment of the invention, the target nucleic acid is an endogenous gene which encodes an immunoglobulin. Immunoglobulin loss can be detected both for cells which secrete immtnoglobulins into the culture medium and for cells in which the immunoglobulin is displayed on the cell surface. Where the immunoglobulin is present on the cell surface, its absence can be identified for individual cells, for example, by FACS analysis, immunofluorescence microscopy or ligand immobilization to a support. In a preferred embodiment, cells can be mixed with antigen-coated magnetic beads which, when sedimented, will remove from the cell suspension all cells having an immunoglobulin of the desired specificity displayed on the surface, leaving candidate hypermutated cells behind.

The technique can be extended to any immunoglobulin molecule sequence, including antibodies, as well as T-cell receptor sequences and the like. The selection of immunoglobulin molecules will depend on the nature of the clonal population of cells which it is desired to assay according to the invention.

Alternatively, as discussed above, cells can be selected by sequencing of target nucleic acids, such as V genes, and detection of mutations by sequence comparison. This process can be automated in order to increase throughput.

In a further embodiment, cells which hypermutate V genes can be detected by assessing change in antigen binding activity in the immunoglobulins produced in a clonal cell population. For example, the quantity of antigen bound by a specific unit amount of cell medium or extract can be assessed in order to determine the proportion of immunoglobulin produced by the cell which retains a specified binding activity. As the V genes are mutated, so binding activity will be varied and the proportion of produced immunoglobulin which binds a specified antigen will be reduced.

Alternatively, cells can be assessed in a similar manner for the ability to develop a novel binding affinity, such as by exposing them to an antigen or mixture of antigens which are initially not bound and observing whether a binding affinity develops as the result of hypermutation.

Cells which target sequence hypermutation are assessed for mutations in other nucleic acid regions to select cells which selectively hypermutate target sequences and which do not substantially mutate non-target sequences (i.e., to identify cells in which non-target sequences mutate at background mutation rates). A convenient region to assay is the constant (C) region of an immunoglobulin gene. C regions are not subject to directed hypermutation according to the invention. The assessment of C regions is preferably made by sequencing and comparison, since this is the most certain method for determining the absence of mutations. However, other techniques can be employed, such as monitoring for the retention of C region activities, for example, by monitoring complement fixation, which can be disrupted by hypermutation events.

Genetic Manipulation of Cells

Hypermutating cells according to the invention can be selected from cells which have been genetically manipulated to enhance rates of hypermutation in the Ig V-region. Genes which are responsible for modulation of mutation rates include, in general, genes involved in nucleic acid repair procedures in the cell. Genes which are manipulated in accordance with the present invention can be up-regulated, down-regulated or deleted.

Up- or down-regulation refers to an increase, or decrease, in activity of the gene product encoded by the gene in question by at least 10%, preferably 25%, more preferably 40, 50, 60, 70, 80, 90, 95, 99% or more. Up-regulation can of course represent an increase in activity of over 100%, such as 200% or 500%. A gene which is 100% down-regulated is functionally deleted and is referred to herein as “deleted”.

Preferred genes manipulated in accordance with the present invention include analogues and/or paralogues of the Rad51 gene, in particular xrcc2, xrcc3 and Rad51b genes.

Rad51 analogues and/or paralogues are advantageously down-regulated, and preferably deleted. Down-regulation or deletion of one or more Rad51 paralogues gives rise to an increase in hypermuitation rates in accordance with the invention. Preferably, two or more Rad51 genes, including analogues and/or paralogues thereof, are down-regulated or deleted.

In a highly preferred embodiment, avian cell lines such as the chicken DT40 cell line are modified by deletion of xrcc2 and/or xrcc3. Δxrcc2 DT40 as well Δxrcc3-DT40 are constitutively hypermutating cell lines isolated in accordance with the present invention. Down-regulated genes can be generated by gene disruption techniques well known in the art (see, e.g., U.S. Pat. No. 6,214,622).

Adaptation of Endogenous Gene Products

Having obtained a cell line which constitutively hypermutates a target gene, such as an immunoglobulin V region gene, the present invention provides for the adaptation of the endogenous gene product, by constitutive hypermutation, to produce a gene product having novel properties. For example, the present invention provides for the production of an immunoglobulin having a novel binding specificity or an altered binding affinity.

The process of hypermutation is employed in nature to generate improved or novel binding specificities in immunoglobulin molecules. Thus, by selecting cells according to the invention which produce immunoglobulins capable of binding to the desired antigen and then propagating these cells in order to allow the generation of further mutants, cells which express immunoglobulins having improved binding to the desired antigen can be isolated.

A variety of selection procedures can be applied for the isolation of mutants having a desired specificity. These include Fluorescence Activated Cell Sorting (FACS), cell separation using magnetic particles, antigen chromatography methods and other cell separation techniques such as use of polystyrene beads, as are known and routine in the art.

Separating cells using magnetic capture can be accomplished by conjugating the antigen of interest to magnetic particles or beads. For example, the antigen can be conjugated to superparamagnetic iron-dextran particles or beads as supplied by Miltenyi Biotec GmbH. These conjugated particles or beads are then mixed with a cell population which can express a diversity of surface immunoglobulins. If a particular cell expresses an immunoglobulin capable of binding the antigen, it will become complexed with the magnetic beads by virtue of this interaction. A magnetic field is then applied to the suspension which immobilizes the magnetic particles, and retains any cells which are associated with them via the covalently linked antigen. Unbound cells which do not become linked to the beads are then washed away, leaving a population of cells which is isolated purely on its ability to bind the antigen of interest. Reagents and kits are available from various sources for performing such one-step isolations, and include Dynal Beads (Dynal AS; http://www.dynal.no), MACS-Magnetic Cell Sorting (Miltenyi Biotec GmbH; http://www.miltenyibiotec.com), CliniMACS (AmCell; http://www.amcell.com) as well as Biomag, Amerlex-M beads and others.

Fluorescence Activated Cell Sorting (FACS) can be used to isolate cells on the basis of their differing surface molecules, for example surface-displayed immunoglobulins. Cells in the sample or population to be sorted are stained with specific fluorescent reagents which bind to the cell surface molecules. These reagents would be the antigen(s) of interest linked (either directly or indirectly) to fluorescent markers such as fluorescein, Texas Red, malachite green, green fluorescent protein (GFP), or any other fluorophore known to those skilled in the art. The cell population is then introduced into the vibrating flow chamber of the FACS machine. The cell stream passing out of the chamber is encased in a sheath of buffer fluid such as PBS (Phosphate Buffered Saline). The stream is illuminated by laser light and each cell is measured for fluorescence, indicating binding of the fluorescent labeled antigen. The vibration in the cell stream causes it to break up into droplets, which carry a small electrical charge. These droplets can be steered by electric deflection plates under computer control to collect different cell populations according to their affinity for the fluorescent labeled antigen. In this manner, cell populations which exhibit different affinities for the antigen(s) of interest can be easily separated from those cells which do not bind the antigen. FACS machines and reagents for use in FACS are widely available from sources world-wide such as Becton-Dickinson, or from service providers such as Arizona Research Laboratories (http://www.arl.arizona.edu/facs/).

Another method which can be used to separate populations of cells according to the affinity of their cell surface protein(s) for a particular antigen is affinity chromatography. In this method, a suitable resin (for example CL-600 Sepharose, Pharmacia Inc.) is covalently linked to the appropriate antigen. This resin is packed into a column, and the mixed population of cells is passed over the column. After a suitable period of incubation (for example 20 minutes), unbound cells are washed away using (for example) PBS buffer. This leaves only that subset of cells expressing immunoglobulins which bound the antigen(s) of interest, and these cells are then eluted from the column using (for example) an excess of the antigen of interest, or by enzymatically or chemically cleaving the antigen from the resin. This can be done using a specific protease such as factor X, thrombin, or other specific protease known to those skilled in the art to cleave the antigen from the column via an appropriate cleavage site which has previously been incorporated into the antigen-resin complex. Alternatively, a non-specific protease, for example trypsin, can be employed to remove the antigen from the resin, thereby releasing that population of cells which exhibited affinity for the antigen of interest.

Insertion of Heterologous Transcription Units

In order to maximize the chances of quickly selecting an antibody variant capable of binding to any given antigen, or to exploit the hypermutation system for non-immunoglobulin genes, a number of techniques can be employed to engineer cells according to the invention such that their hypermutating abilities can be exploited.

In a first embodiment, transgenes are transfected into a cell according to the invention such that the transgenes become targets for the directed hypermutation events. The plasmids used for delivering the transgene to the cells are of conventional construction and comprise a coding sequence encoding the desired gene product under the control of a promoter. Gene transcription from vectors in cells according to the invention can be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter, or from a very strong promoter, e.g., a ribosomal protein promoter. The promoter normally associated with the heterologous coding sequence also can be used provided it is compatible with the host system of the invention.

Transcription of a heterologous coding sequence by cells according to the invention can be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer can be spliced into the vector at a position 5′ or 3′ to the coding sequence, but is preferably located at a site 5′ from the promoter.

Advantageously, a eukaryotic expression vector can comprise a locus control region (LCR). LCRs are capable of directing high-level integration site independent expression of transgenes integrated into host cell chromatin, which is of importance especially where the heterologous coding sequence is to be expressed in the context of a permanently-transfected eukaryotic cell line in which chromosomal integration of the vector has occurred, in vectors designed for gene therapy applications or in transgenic animals. For example, one such locus control region is located about 50 kilobases upstream of the human β globin gene (see, e.g., Tuan et al., 1985, Proc. Natl. Acad. Sci. USA, 83: 1359-1363; WO 89/01517; Behringer, et al., 1989, Science, 245: 971-973; Enver, et al., 1989, Proc. Natl. Acad. Sci. USA, 86: 7033-7037; Hanscombe, et al., 1989, Genes Dev., 3: 1572-1581; Van Assendelft, et al., 1989, Cell, 56: 967-977; and Grosveld, et al, 1987, Cell 51: 975-985, the entireties of which are incorporated by reference herein).

Eukaryotic expression vectors will also contain sequences necessary for the termination of transcription and for stabilizing the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA.

An expression vector includes any vector capable of expressing a coding sequence encoding a desired gene product that is operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding a heterologous coding sequence can be inserted into a vector suitable for expression of cDNAs in mammalian cells, e.g., a CMV enhancer-based vector such as pEVRF (Matthias, et al., 1989,NAR 17: 6418).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion (e.g., by restriction fragment analysis and/or sequencing). Suitable methods for constructing expression vectors, preparing in vitro transcripts, introducing DNA into host cells, and performing analyses for assessing gene product expression and function are known to those skilled in the art. Gene presence, amplification and/or expression can be measured in a sample directly, for example, by conventional Southern blotting, by Northern blotting to quantitate the transcription of RNA, by dot blotting (DNA or RNA analysis), or by in situ hybridization, using an appropriately labeled probe which can be based on a sequence provided herein. Those skilled in the art will readily envisage how these methods can be modified, if desired.

In one variation of the first embodiment, transgenes according to the invention comprise sequences which direct hypermutation. Such sequences have been characterized, and include those sequences set forth in Klix et al, 1998, Eur. J. Immunol. 28: 317-326, and Sharpe et al, 1991, EMBO J. 10: 2139-2145, incorporated herein by reference. Thus, an entire locus capable of expressing a gene product and directing hypermutation to the transcription unit encoding the gene product is transferred into the cells. The transcription unit and the sequences which direct hypermutation are thus exogenous to the cell. However, although exogenous, the sequences which direct hypermutation themselves can be similar or identical to the sequences which direct hypermutation naturally found in the cell.

In a second embodiment, the endogenous V gene(s) or segments thereof can be replaced with heterologous V gene(s) by homologous recombination, or by gene targeting using, for example, a Lox/Cre system or an analogous technology or by insertion into hypermutating cell lines which have spontaneously deleted endogenous V genes. Alternatively, V region gene(s) can be replaced by exploiting the observation that hypermutation is accompanied by double stranded breaks in the vicinity of rearranged V genes.

### EXAMPLES

The invention is further described below, for the purposes of illustration only, in the following examples.

### Example 1

### Selection of a Hypermutating Cell

In order to screen for a cell that undergoes hypermutation in vitro, the extent of diversity that accumulates in several human Burkitt lymphomas during clonal expansion is assessed. The Burkitt lines BL2, BL41 and BL70 are kindly provided by G. Lenoir (IARC, Lyon, France) and Ramos (Klein et al., 1975, Intervirology 5: 319-334) is provided by D. Fearon (Cambridge, UK). Their rearranged V_{H} genes are PCR amplified from genomic DNA using multiple V_{H} family primers together with a J_{H} consensus oligonucleotide. Amplification of rearranged V_{H} segments is accomplished using Pfu polymerase together with one of 14 primers designed for each of the major human V_{H} families (Tomlinson, 1997, V Base database of human antibody genes. Medical Research Council, Centre for Protein Engineering, UK. http://www.mrc-cpe.cam.ac.uk/) and a consensus J_{H} back primer which anneals to all six human J_{H} segments (JOL48, 5′-GCGGTACCTGAGGAGACGGTGACC-3′, gift of C. Jolly). Amplification of the Ramos V_{H} from genomic DNA is performed with oligonucleotides RVHFOR (5′-CCCCAAGCTTCCCAGGTGCAGCTACAGCAG) and JOL48. Amplification of the expressed V_{H}-Cμ cDNA is performed using RVHFOR and Cμ 2BACK (5′-CCCCGGTACCAGATGAGCTTGGACTTGCGG). The genomic CμC1/2 region is amplified using Cμ2BACK with Cμ1FOR (5′-CCCCAAGCTTCGGGAGTGCATCCGCCCCAACCCTT); the functional Cμ allele of Ramos contains a C at nucleotide 8 of Cμ2 as opposed to T on the non-functional allele. Rearranged V_{λ}'s are amplified using 5′-CCCCAAGCTTCCCAGTCTGCCCTGACTCAG and 5′-CCCCTCTAGACCACCTAGGACGGTC-AGCTT. PCR products are purified using QIAquick (Qiagen) spin columns and sequenced using an ABI377 sequencer following cloning into M13. Mutations are computed using the GAP4 alignment program (Bonfield et al., 1995, NAR 23: 4992-99.).

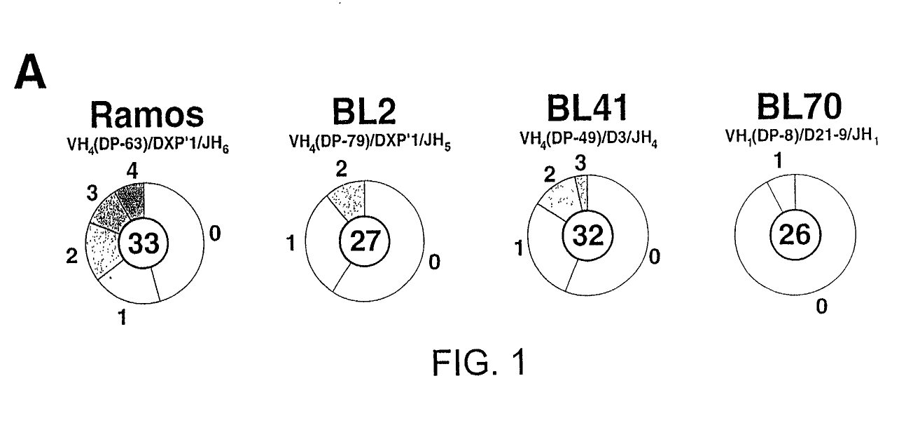

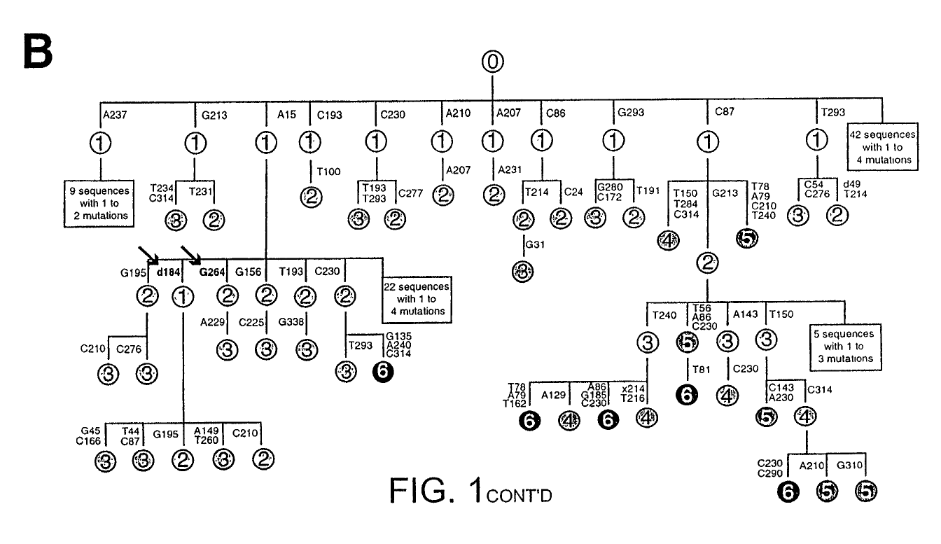

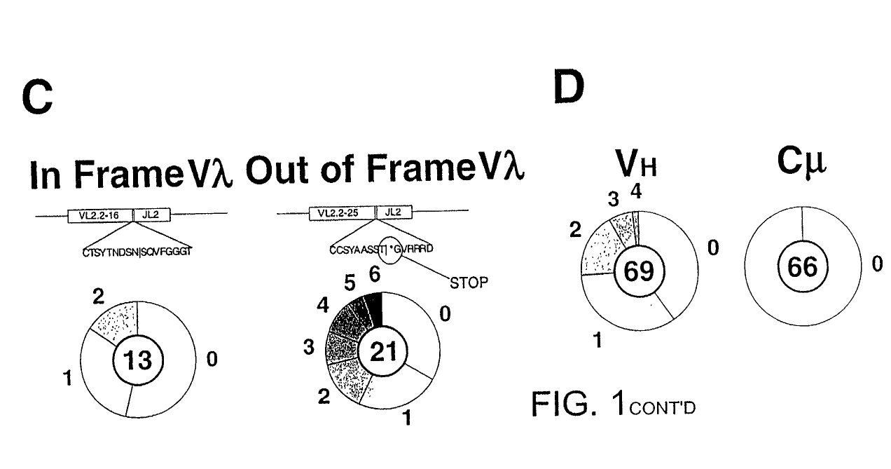

Sequencing of cloned PCR products reveals considerable diversity in the Ramos cell line (a prevalence of 2.8×10³ mutations bp¹ in the V_{H}) although significant heterogeneity is also observed in BL41 as well as in BL2. See FIG. 1A. Sequence diversity in the rearranged V_{H} genes of four sporadic Burkitt lymphoma lines are shown as pie charts. The rearranged V_{H} genes in each cell line are PCR amplified and cloned into M13. For each cell line, the consensus is taken as the sequence common to the greatest number of M13 clones and a germline counterpart (indicated above each pie) assigned on the basis of closest match using the VBASE database of human immunoglobulin sequences (Tomlinson, 1997, supra). The V_{H} consensus sequence for Ramos used herein differs in 3 positions from the sequence determined by Chapman et al., 1995, Blood 85: 2176-2181; Chapman, 1994, Curr. Op. Struct. Biol. 4: 618-622, five positions from that determined by Ratech, 1992, Biochem. Biophys. Res. Commun. 182: 1260-1263 and six positions from its closest germline counterpart V_{H}4(DP63).

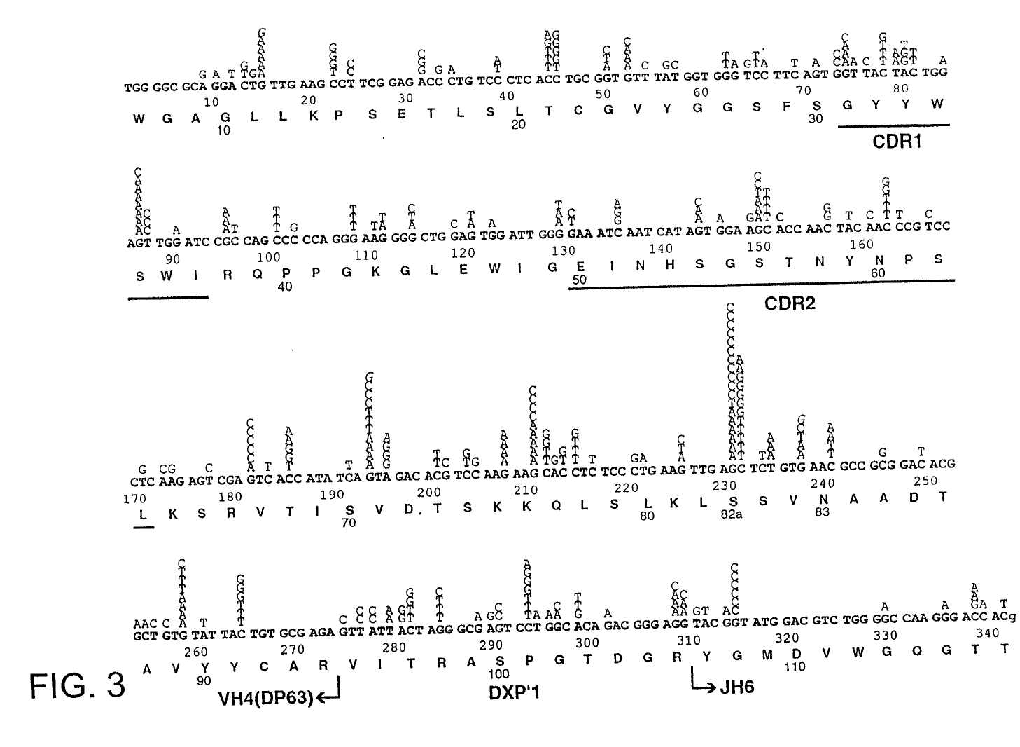

The analysis of V_{H} diversity in Ramos is extended by sequencing the products from nine independent PCR amplifications. This enables a likely dynastic relationship between the mutated clones in the population to be deduced, minimizing the number of presumed independent repeats of individual nucleotide substitutions (FIG. 1B). 315 M13V_{H} clones obtained from nine independent PCR amplifications are sequenced; the dynasty only includes sequences identified (rather than presumed intermediates). Individual mutations are designated according to the format “C230” with 230 being the nucleotide position in the Ramos V_{H} (numbered as in FIG. 3) and the “C” indicating the novel base at that position. The criterion used to deduce the genealogy is a minimization of the number of independent occurrences of the same nucleotide substitution. The majority of branches contain individual members contributed by distinct PCR amplifications. The rare deletions and duplications are indicated by the prefix “x” and “d” respectively. Arrows highlight two mutations (a substitution at position 264 yielding a stop codon and a duplication at position 184) whose position within the tree implies that mutations can continue to accumulate following loss of functional heavy chain expression.

PCR artifacts make little contribution to the database of mutations. Not only is the prevalence of nucleotide substitutions greatly in excess of that observed in control PCR amplifications (<0.05×10⁻³ bp⁻) but also identically mutated clones (as well as dynastically related ones) are found in independent amplifications. In many cases, generations within a lineage differ by a single nucleotide substitution indicating that only a small number of substitutions have been introduced in each round of mutation.

Analysis of Vλ rearrangements reveals that Ramos harbors an in-frame rearrangement of Vλ2.2-16 (as described by Chapman et al., 1995, supra) and an out-of-frame rearrangement of Vλ2.2-25. There is mutational diversity in both rearranged V_{λ}'s although greater diversity has accumulated in the non-functional allele (FIG. 1C).

A classic feature of antibody hypermutation is that mutations largely accumulate in the V region but scarcely in the C region. This is also evident in the mutations that have accumulated in the Ramos Ig_{H} locus (FIG. 1D). M13 clones containing cDNA inserts extending through V_{H}, Cμ1 and the first 87 nucleotides Cμ2 are generated by PCR from the initial Ramos culture. The Pie charts (presented as in FIG. 1A) depict the extent of mutation identified in the 341 nucleotide stretch of V_{H} as compared to a 380 nucleotide stretch of Cμ extending from the beginning of Cμ1.

The IgM immunoglobulin produced by Ramos is present both on the surface of the cells and, in secreted form, in the culture medium. Analysis of the culture medium reveals that Ramos secretes immunoglobulin molecules to a very high concentration, approximately 1 μg/ml. Thus, Ramos is capable of secreting immunoglobulins to a level which renders it unnecessary to reclone immunoglobulin genes into expression cell lines or bacteria for production.

### Example 2

### V_{H} diversification in Ramos is Constitutive

To address whether V gene diversification is ongoing, the cells are cloned and V_{H} diversity assessed using a MutS-based assay after periods of in vitro culture. The Ramos V_{H} is PCR-amplified and purified as described above using oligonucleotides containing a biotinylated base at the 5′-end. Following denaturation/renaturation (99° C. for 3 min.; 75° C. for 90 min.), the extent of mutation is assessed by monitoring the binding of the mismatched heteroduplexed material to the bacterial mismatch-repair protein MutS using a solid phase assay as described above (Jolly et al., supra). Binding of heteroduplexed nucleic acids to MutS is detected using ECL. to the detect the presence of the reporter enzyme.

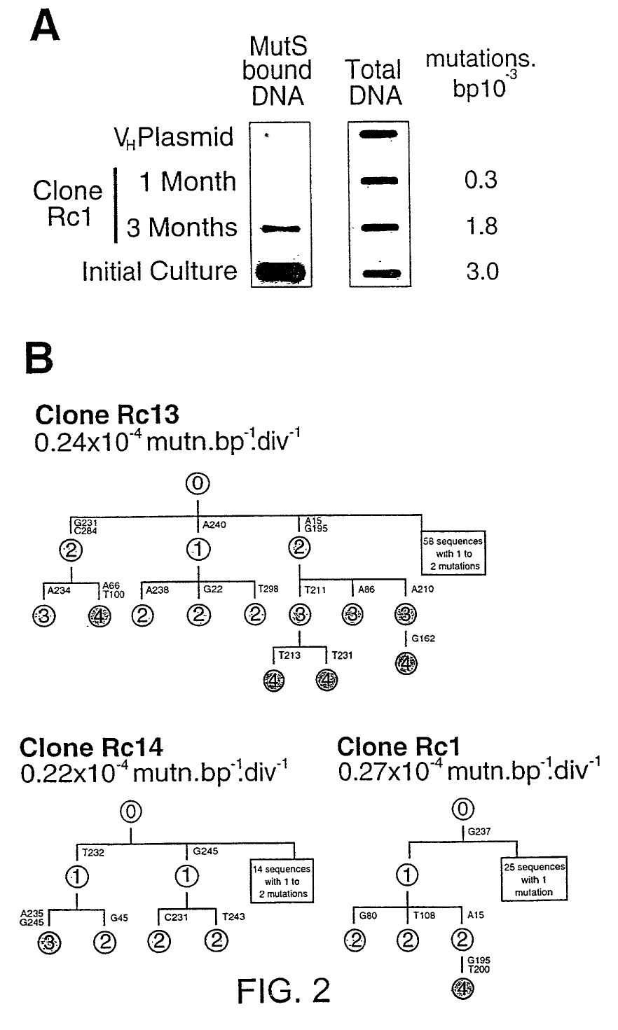

The results indicate that V_{H} diversification is indeed ongoing (see, FIG. 2A). DNA is extracted from Ramos cells that have been cultured for 1 or 3 months following limit dilution cloning. The rearranged V_{H} is PCR amplified using biotinylated oligonucleotides prior to undergoing denaturation/renaturation; mismatched heteroduplexes are then detected by binding to immobilized MutS as previously described (Jolly et al., supra). An aliquot of the renatured DNA is bound directly onto membranes to confirm matched DNA loading (Total DNA control). Assays performed on the Ramos V_{H} amplified from a bacterial plasmid template as well as from the initial Ramos culture are included for comparison.

The V_{H} genes are PCR-amplified from Ramos cultures that have been expanded for four (Rc1) or six (Rc13 and 14) weeks (FIG. 2B). A mutation rate for each clone is indicated and is calculated by dividing the prevalence of independent V_{H} mutations at 4 or 6 weeks post-cloning by the presumed number of cell divisions based on a generation time of 24 h. The sequences reveal step-wise mutation accumulation with a mutation rate of about 0.24×10⁻⁴ mutations bp⁻¹ generation⁻¹.

Direct comparison of the V_{H} mutation rate in Ramos to that in other cell-lines is not straightforward since there is little information on mutation rates in other lines as judged by unselected mutations incorporated throughout the V_{H} obtained following clonal expansion from a single precursor cell. However, the prevalence of mutations following a two-week expansion of 50 precursor BL2 cells has been determined under conditions of mutation induction (2.7×10⁻³ mutations bp⁻¹; see, e.g., Denepoux et al., 1997, Immunity 6: 35-46). Similar experiments performed with Ramos under conditions of normal culture reveal a mutation prevalence of 2.3×10⁻³ mutations bp⁻¹. Various attempts to enhance the mutation rate by provision of cytokines, helper T cells etc., have proven unsuccessful. Thus, the rate of mutation that can be achieved by specific induction in BL2 cells appears to be similar to the constitutive rate of V_{H} mutation in Ramos.

### Example 3

### Examination of the Nature of V_{H} Mutations in Ramos

A database of mutational events is created which combines those detected in the initial Ramos culture (from 141 distinct sequences) with those detected in four subclones that have been cultured in various experiments without specific selection (from a further 135 distinct sequences). This database is created after the individual sets of sequences have been assembled into dynastic relationships (as detailed in the legend to FIG. 1B) to ensure that clonal expansion of an individual mutated cell does not lead to a specific mutational event being counted multiple times. Here an analysis of this composite database of 340 distinct and presumably unselected mutational events (200 contributed by the initial Ramos culture and 140 from the expanded subclones) is described; separate analysis of the initial and subclone populations yields identical conclusions.

The overwhelming majority of the mutations (333 out of 340) are single nucleotide substitutions. A small number of deletions (4) and duplications (3) are observed but no untemplated insertions; these events are further discussed below. There are only five sequences which exhibited nucleotide substitutions in adjacent positions; however, in three of these five cases, the genealogy revealed that the adjacent substitutions have been sequentially incorporated. Thus, the simultaneous creation of nucleotide substitutions in adjacent positions is a rare event.

The distribution of the mutations along the V_{H} is highly non-random (See FIG. 3). Independently occurring base substitutions are indicated at each nucleotide position. The locations of CDR1 and 2 are indicated. Nucleotide positions are numbered from the 3′-end of the sequencing primer with nucleotide position +1 corresponding to the first base of codon 7; codons are numbered according to Kabat (Kabat et al, 1991, In Sequences of Proteins of Immunological Interest, 5th edition, Bethesda, Md.:NIH vol. 1, pp. 669, 671, 687, 696). Mutations indicated in italics (nucleotide position 15, 193, 195 and 237) are substitutions that occur in a mutated subclone and have reverted the sequence at that position to the indicated consensus.

The major hotspot is at the G and C nucleotides of the Ser82a codon, which has previously been identified as a major intrinsic mutational hotspot in other V_{H} genes (Wagner et al., 1995, Nature 376: 732; Jolly et al., 1996, Semin. Immunol. 8: 159-168.) and conforms to the RGYW consensus (Rogozin and Kolchanov, 1992, Biochem. Biophys. Acta 1171: 11-18; Betz et al., 1993, Immunol. Today 14: 405-411). While the dominant intrinsic mutational hotspot in many V_{H} genes is at Ser31, this codon is not present in the Ramos consensus V_{H} (or its germline counterpart) which have Gly at that position. The individual nucleotide substitutions show a marked bias in favor of transitions (51% rather than randomly-expected 33%). There is also a striking preference for targeting G and C which account for 82% of the nucleotides targeted (Table 1).

TABLE 1
Nucleotide Substitution Preferences Of Hypermutation In Ramos

| Parental | Frequency of substitution to: | | | | |
|---|---|---|---|---|---|
| Nucleotide | T | C | G | A | Total |
| T | — | 3.9 | 1.2 | 3.0 | 8.1 |
| C | 17.4 | — | 12.6 | 4.8 | 34.8 |
| G | 7.2 | 15.9 | — | 24.0 | 47.1 |
| A | 2.4 | 1.8 | 5.7 | — | 9.9 |

Single nucleotide substitutions were computed on the V_{H} coding strand and are given as the percentage of the total number (333) of independent, unselected nucleotide substitutions identified.

### Example 4

### Selection of Hypermutating Cells by IgM-Loss

Analysis of the Ramos variants reveals several mutations that must have inactivated V_{H} (see FIG. 1B) suggesting it might be possible for the cells to lose IgM expression but remain viable. If this is the case, Ig expression loss would be an easy means to select a constitutively hypermutating B cell line.

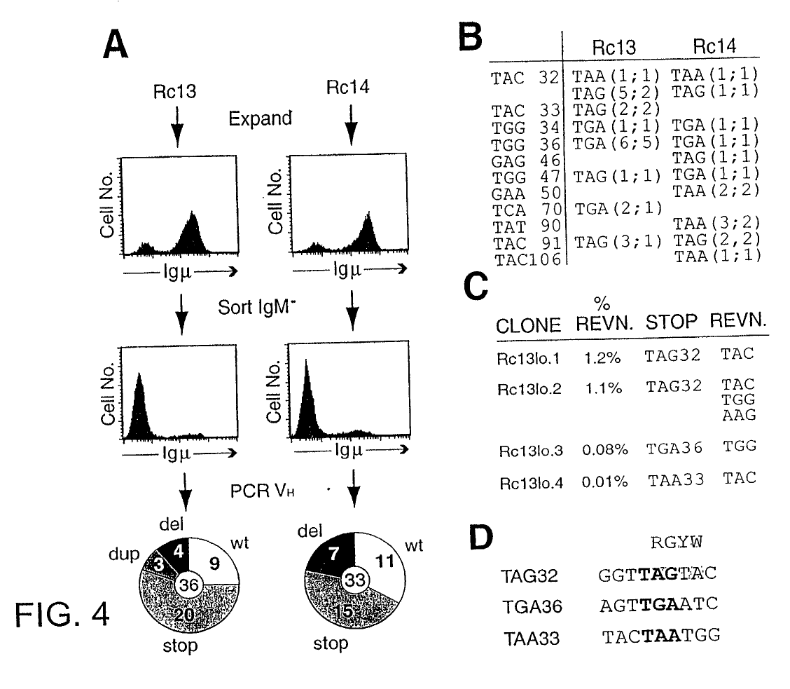

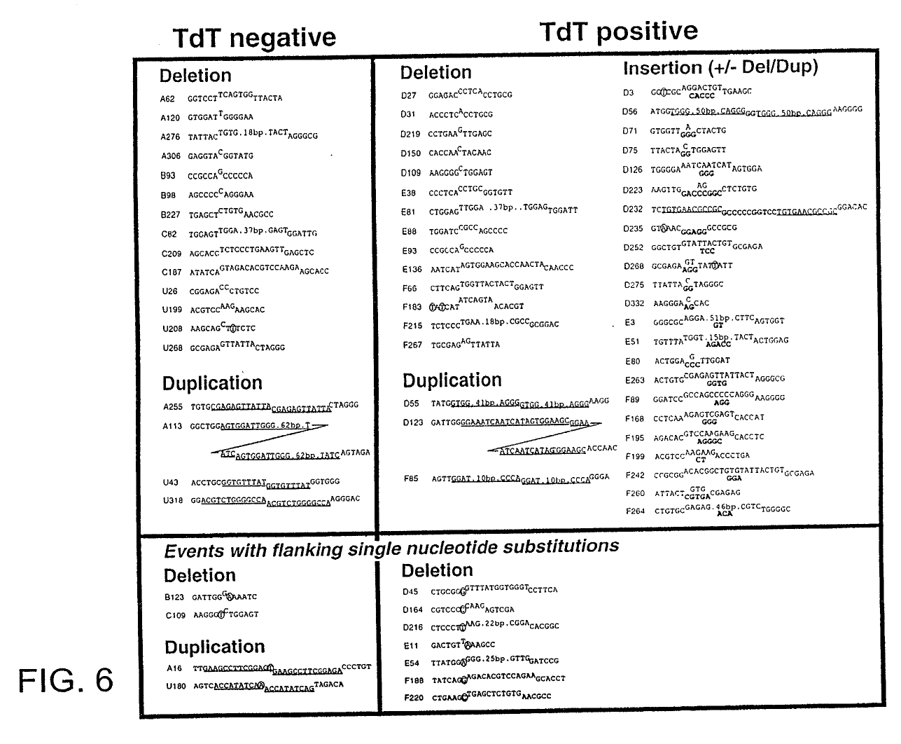

Analysis of the Ramos culture reveals it to contain 8% surface IgM⁻ cells. Such IgM⁻ loss variants are generated during in vitro culture, as follows. The starting Ramos culture is transfected with a pSV2neo plasmid, diluted into 96-well plates and clones growing in selective medium allowed to expand. Flow cytometry performed on the expanded clones six months after the original transfection reveals the presence of IgM-loss variants, constituting 16% and 18% of the two clonal populations (Rc13 and Rc14) shown here (FIG. 4A). Enrichment by a single round of sorting yields subpopulations that contain 87% (Rc13) and 76% (Rc14) surface IgM-negative cells. Following PCR amplification of the rearranged V_{H} gene in these subpopulations, sequencing reveals that 75% (Rc13) and 67% (Rc14) of the cloned V_{H} segments contained a nonsense (stop), deletion (del) or duplication (dup) mutation within the 341 nucleotide V_{H} stretch analyzed. The remainder of the clones are designated wild-type (wt) although no attempt is made to discriminate possible V_{H}-inactivating missense mutations. The 4 deletions and 3 duplications identified in the Rc13 population are all distinct whereas only 4 distinct mutations account for the 7 Rc14 sequences determined that harbor deletions. The nature of the deletions and duplications is presented in FIG. 6: each event is named with a letter followed by a number. The letter gives the provenance of the mutation (A, B and C being the cloned TdT⁻ control transfectants, D, E and F the TdT⁺ transfectants and U signifies events identified in the initial, unselected Ramos culture); the number indicates the first nucleotide position in the sequence string. Nucleotides deleted are specified above the line and nucleotides added (duplications or non-templated insertions) below the line; single nucleotide substitutions are encircled with the novel base being specified. The duplicated segments of V_{H} origin are underlined; non-templated insertions are in bold. With several deletions or duplications, the event is flanked by a single nucleotide of unknown provenance. Such flanking changes could well arise by nucleotide substitution (rather than by non-templated insertion) and these events therefore separately grouped; the assignment of the single base substitution (encircled) to one or other end of the deletion/duplication is often arbitrary.

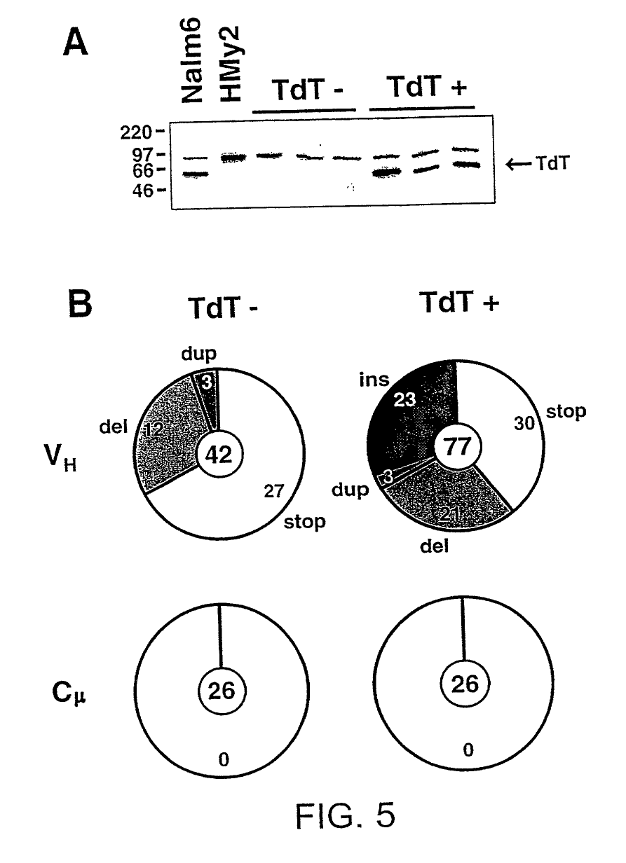

The IgM⁻ cells are enriched in a single round of sorting prior to PCR amplification and cloning of their V_{H} segments. The sequences reveal a considerable range of V_{H}-inactivating mutations (stop codons or frameshifts) (FIG. 4) although diverse inactivating mutations are even evident in IgM-loss variants sorted after only 6 weeks of clonal expansion (see FIG. 5). In FIG. 5A expression of TdT in three pSV-pβG/TdT and three control transfectants of Ramos is compared by Western blot analysis of nuclear protein extracts. Nalm6 (a TdT-positive human pre-B cell lymphoma) and HMy2 (a TdT-negative mature human B lymphoma) provided controls.

In FIG. 5B, pie charts are shown depicting independent mutational events giving rise to IgM-loss variants. IgM⁻ variants (constituting 1-5% of the population) are obtained by sorting the three TdT⁺ and three TdT⁻ control transfectants that have been cultured for 6 weeks following cloning. The V_{H} regions in the sorted subpopulations are PCR amplified and sequenced. The pie charts depict the types of mutation giving rise to V_{H} inactivation with the data obtained from the TdT⁺ and TdT⁻ IgM⁻ subpopulations separately pooled. Abbreviations are as in FIG. 4A except that “ins” indicates clones containing apparently non-templated nucleotide insertions. Clones containing deletions or duplications together with multiple nucleotide non-templated insertions are only included within the “ins” segment of the pie. Only unambiguously distinct mutational events are computed. Thus, of the 77 distinct V_{H} inactivating mutations identified in the TdT⁺ IgM-loss subpopulations, 30 distinct stop codon mutations are identified; if the same stop codon have been independently created within the IgM-loss population derived from a single Ramos transfectant, this would have been underscored.

The stop codons are created at variety of positions (FIG. 4B) but are not randomly located. FIG. 4B summarizes the nature of the stop codons observed in the Rc13 and Rc14 IgM-loss populations. At least eight independent mutational events yield the nonsense mutations which account for 20 out of the 27 non-functional V_{H} sequences in the Rc13 database; a minimum of ten independent mutational events yield the nonsense mutations which account for 15 of the 22 non-functional V_{H} sequences in the Rc14 database. The numbers in parentheses after each stop codon give the number of sequences in that database that carry the relevant stop codon followed by the number of these sequences that are distinct, as discriminated on the basis of additional mutations. Analysis of stop codons in IgM-loss variants selected from four other clonal populations reveals stop codon creation at a further five locations within V_{H}. In data obtained in six independent experiments, stop codon creation is restricted to 16 of the 39 possible sites; the DNA sequences at these preferred sites being biased (on either coding or non-coding strand) towards the RGYW consensus.

Not surprisingly, whereas deletions and insertions account for only a small proportion of the mutations in unselected Ramos cultures (see above), they make a much greater contribution when attention is focused on V_{H}-inactivating mutations. It is notable that a large proportion of the IgM-loss variants can be accounted for by stop-codon/frameshift mutations in the V_{H} itself. This further supports the proposal that hypermutation in Ramos is preferentially targeted to the immunoglobulin V domain—certainly rather than the C domain or, indeed other genes (such as the Igα/Igβ sheath) whose mutation could lead to a surface IgM⁻ phenotype. It also can well be that the Ramos V_{H} is more frequently targeted for hypermutation than its productively rearranged V_{λ}, a conclusion supported by the pattern of mutations in the initial culture (FIG. 1C).

Selection of cells by detection of Ig loss variants is particularly useful where those variants are capable of reverting, i.e. of reaquiring their endogenous Ig-expressing ability. The dynasty established earlier (FIG. 1B) suggests not only that IgM-loss cells could arise but also that they might undergo further mutation. To confirm this, IgM-loss variants sorted from Rc13 are cloned by limiting dilution. Three weeks after cloning, the presence of IgM⁺ revertants in the IgM⁻ subclones is screened by cytoplasmic immunofluorescence analysis of 5×10⁴ cells; their prevalence is given (FIG. 4C). These IgM⁺ revertants are then enriched in a single round of sorting and the V_{H} sequences of the clonal IgM⁻ variant compared to that it of its IgM⁺ revertant descendants.

Cytoplasmic immunofluorescence of ten expanded clonal populations reveals the presence of IgM⁺ revertants at varying prevalence (from 0.005% to 1.2%; FIG. 4C) allowing a mutation rate of 1×10⁻⁴ mutations bp⁻¹ generation⁻¹ to be calculated by fluctuation analysis. This is somewhat greater than the rate calculated by direct analysis of unselected mutations (0.25×10⁻⁴ mutations bp⁻¹ generation⁻¹; see above), probably in part reflecting that different IgM-loss clones revert at different rates depending upon the nature of the disrupting mutation. Indeed, the sequence surrounding the stop codons in the IgM-loss derivatives of Rc13 reveals that TAG32 conforms well to the RGYW consensus (R=purine, Y=pyrimidine and W=A or T; Rogozin and Kolchanov, 1992, supra) which accounts for a large proportion of intrinsic mutational hotspots (Betz et al., 1993, supra) whereas TAA33 and TGA36 do not (FIG. 4D).

### Example 5

### Selection of a Novel Ig Binding Activity

In experiments designed to demonstrate development of novel binding affinities, it is noted that most members of the Ramos cell line described below express a membrane IgM molecule which binds anti-idiotype antibodies (anti-Id1 and anti-Id2), specifically raised against the Ramos surface IgM. However, a few cells retain a surface IgM, yet fail to bind the anti-idiotype antibody. This is due to an alteration in binding affinity in the surface IgM molecule, such that it no longer binds antibody. Cells which express a surface IgM yet cannot bind antibody can be selected in a single round of cell sorting according to the invention.

This is demonstrated by isolating μ positive/id-negative clones which have lost the capacity to bind to anti-Id2 despite the retention of a surface IgM, by ELISA. The clones are sequenced and in six independent clones a conserved V_{H} residue, K70, is found to be mutated to N, M or R as follows:

| Clone | Mutation | |
|---|---|---|
| 2 | K70N | AAG-AAC |
| | S77N | AGC-AAC |
| 4 | K70M | AAG-ATG |
| 9 | S59R | AGT-AGG |
| | K70N | AAG-AAC |
| 10 | K70N | AAG-AAC |
| 12 | K70N | AAG-AAC |
| 13 | K70R | AAG-AGG |

No mutations were observed in the light chain. Thus, it is apparent that mutants can be selected from the Ramos cell line in which the Ig molecule produced has a single base-pair variation with respect to the parent clone.

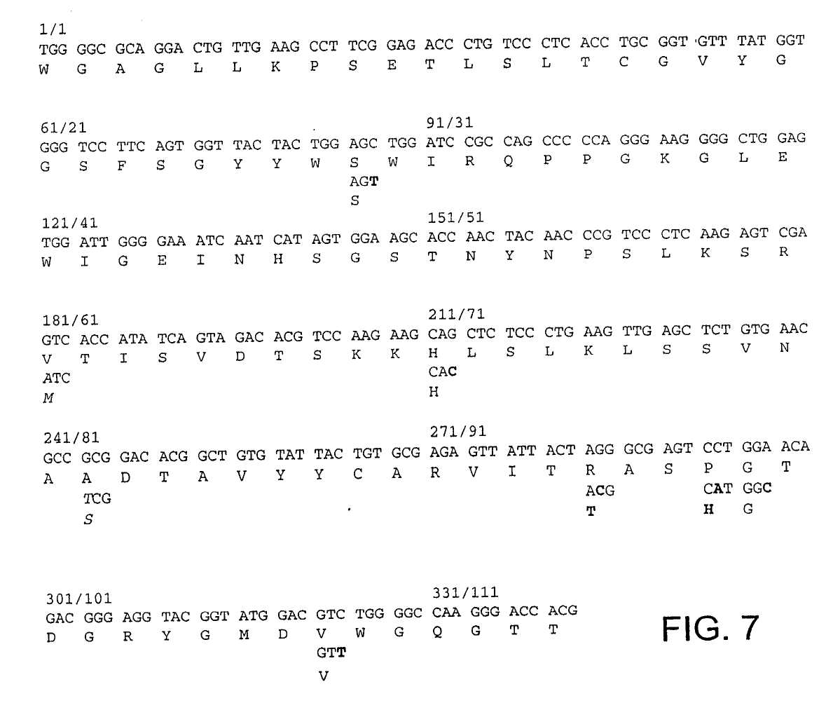

Making use of an anti-Id1, a similar population of cells is isolated which retain expression of the Igμ constant region but which have lost binding to the anti-idiotype antibody. These cells are enriched by sorting cytometry and the sequence of V_{H} determined (FIG. 7). This reveals six mutations when compared with the consensus sequence of the starting population. Two of these mutations result in amino acid sequence changes around CDR3 (R->T at 95 and P->H at 98). Thus, selection of more subtle changes in the immunoglobulin molecule are selectable by assaying for loss of binding.

In further experiments, hypermutating cells according to the invention are washed, resuspended in PBS/BSA (10⁸ cells in 0.25 ml) and mixed with an equal volume of PBS/BSA containing 10% (v/v) antigen-coated magnetic beads. In the present experiment, streptavidin coated magnetic beads (Dynal) are used. After mixing at 4° C. on a roller for 30 minutes, the beads are washed three times with PBS/BSA, each time bringing down the beads with a magnet and removing unbound cells. remaining cells are then seeded onto 96 well plates and expanded up to 10⁸ cells before undergoing a further round of selection. Multiple rounds of cell expansion (accompanied by constitutively-ongoing hypermutation) and selection are performed. After multiple rounds of selection, the proportion of cells which bind to the beads, which is initially at or close to background levels of 0.02%, begins to rise.

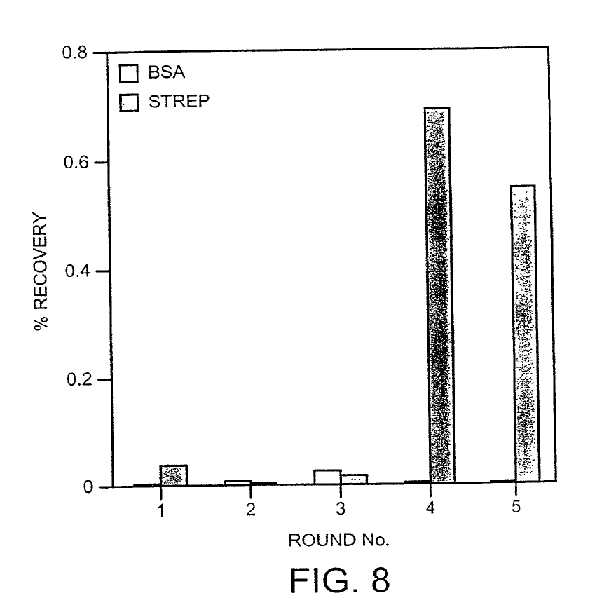

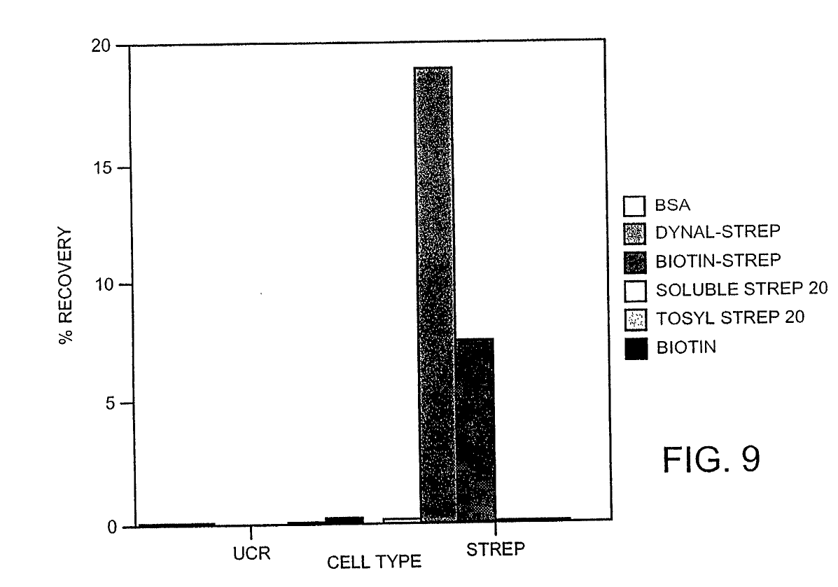

After 4 rounds, enrichment of streptavidin binding cells is seen. This is repeated on the fifth round (FIG. 8). The low percentage recovery reflects saturation of the beads with cells since changing the cell:bead ratio from vast excess to 1:2 allows a recovery of approximately 20% from round five streptavidin binding cells (FIG. 9). This demonstrates successful selection of a novel binding specificity from the hypermutating Ramos cell line, by four rounds of iterative selection.

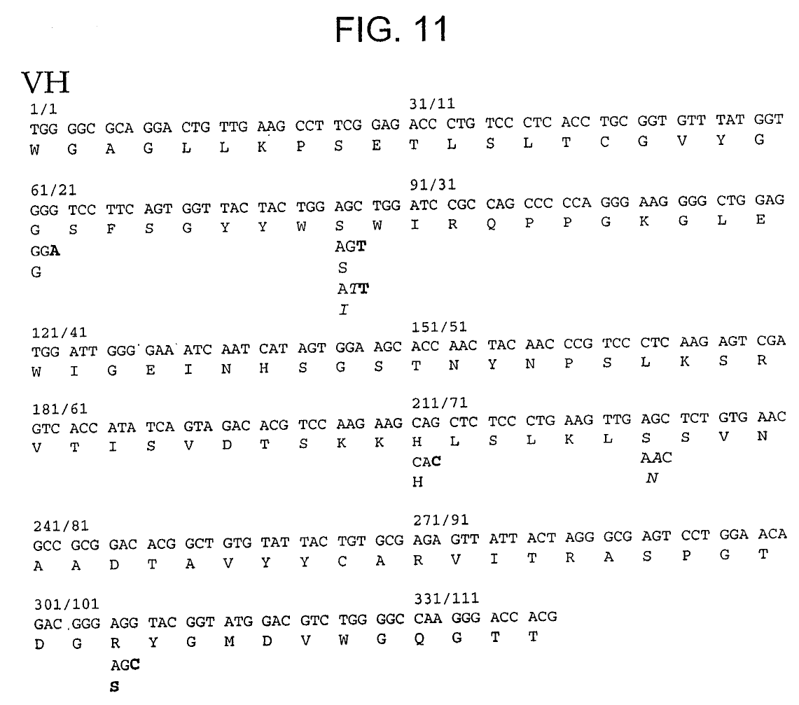

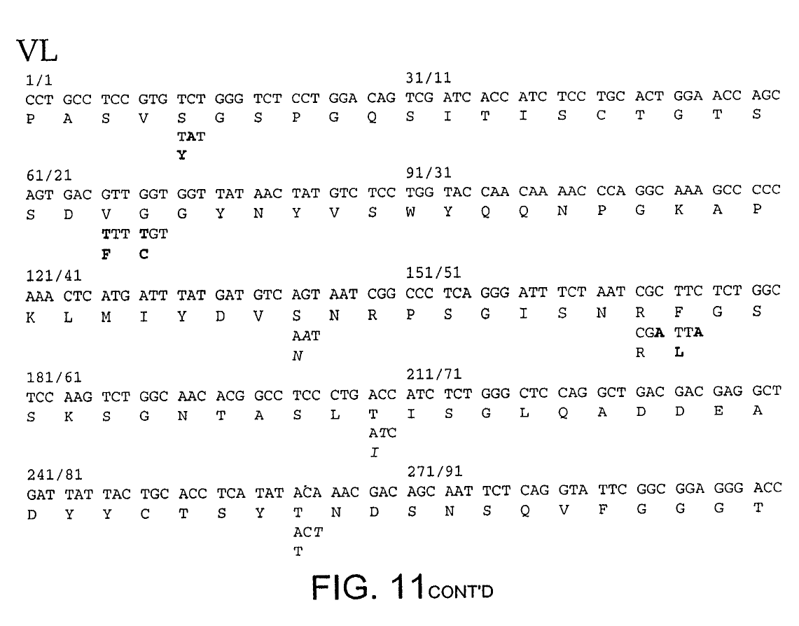

Nucleotide sequencing of the heavy and light chains from the streptavidin binding cells predicts one amino acid change in V_{H} CDR3 and four changes in V_{L} (1 in FR1, 2 in CDR1 and 1 in CDR2) when compared with the consensus sequence of the starting population (FIG. 11).

To ensure that the binding of streptavidin is dependent on expression of surface imnmunoglobulin, immunoglobulin negative variants of the streptavidin binding cells are enriched by sorting cytometry. This markedly reduces the recovery of streptavidin binding cells with an excess of beads. The cells recovered by the Dynal-streptavidin beads from the sorted negative cells are in fact Igμ positive and most likely represent efficient recovery of Igμ streptavidin binding cells contaminating the immunoglobulin negative sorted cell population.

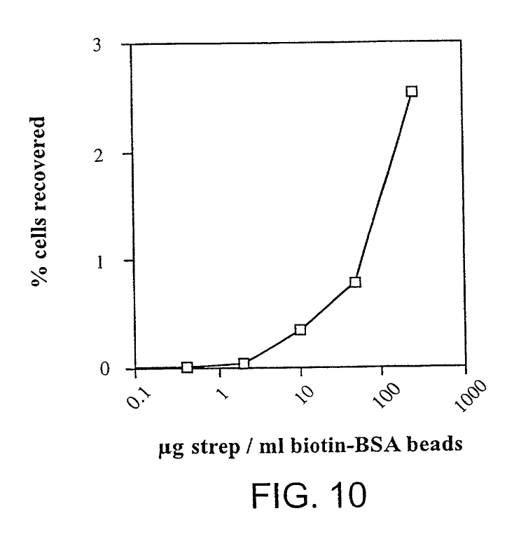

Preliminary data suggest that the efficiency of recovery is reduced as the concentration of streptavidin on the beads is reduced (FIG. 9). This is confirmed by assaying the recovery of streptavidin binding cells with beads incubated with a range of concentrations of streptavidin (FIG. 10). The percentage of cells recoverable from a binding population is dictated by the ratio of beads to cells. In this experiment the ratio is <1: 1 beads:cells.

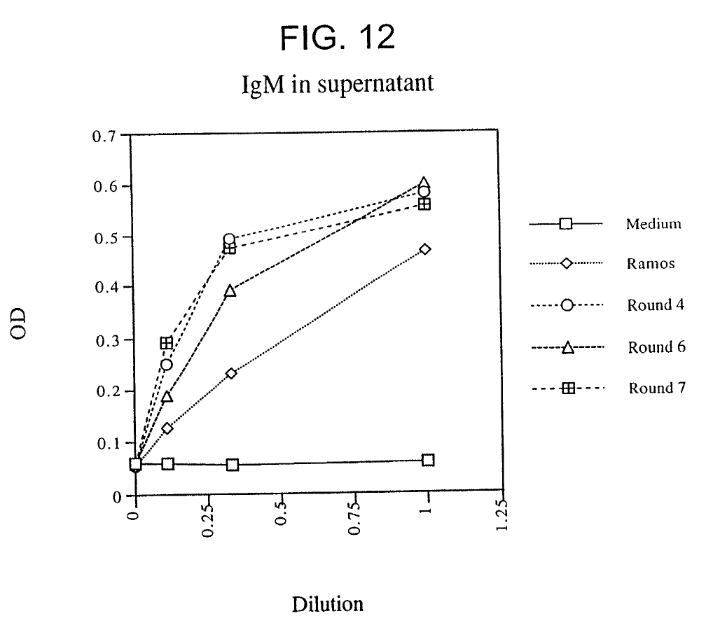

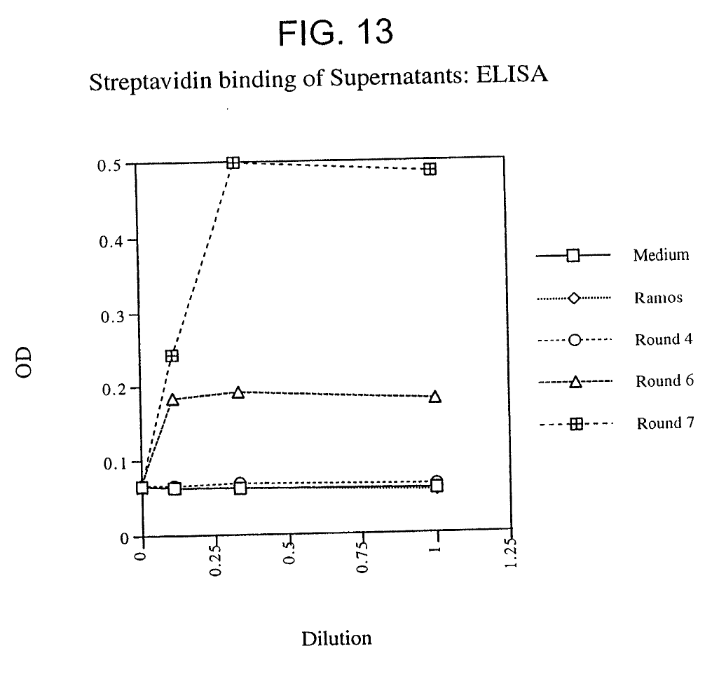

In a further series of experiments, a further two rounds of selection are completed, taking the total to 7. This is accomplished by reducing the concentration of streptavidin bound to the beads from 50 μg/ml in round 5 to 10 μg/ml in round 7. Although the secretion levels of IgM is comparable for the populations selected in rounds 4 to 7 (FIG. 12), streptavidin binding as assessed by ELISA is clearly greatly increased in rounds 6 and 7, in comparison with round 4 (FIG. 13).

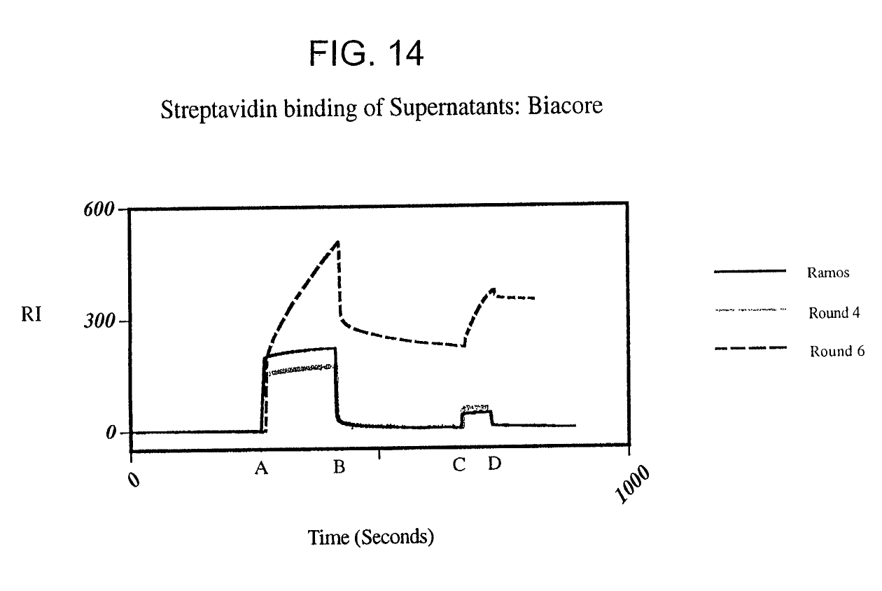

This is confirmed by assessment of binding by Surface Plasmon Resonance on a BiaCore chip coated with streptavidin (FIG. 14). The supernatant from round 7 is injected to flow across the chip at point A, and stopped at point B. At point C, anti-human IgM is injected, to demonstrate that the material bound to the streptavidin is IgM. The gradient A-B represents the association constant, and the gradient B-C to dissociation constant. From the BiaCore trace it is evident that round 6 supernatant displays superior binding characteristics to that isolated from round 4 populations or unselected Ramos cells.

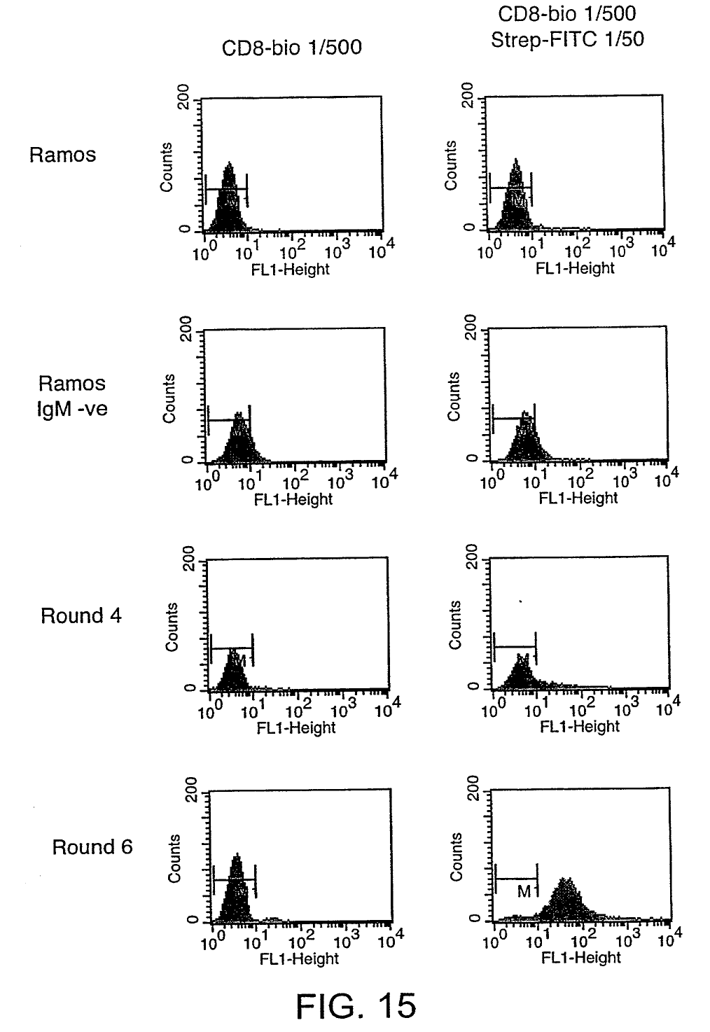

Antibodies from round 6 of the selection process also show improved binding with respect to round 4. Binding of cells from round 6 selections to streptavidin-FITC aggregates, formed by preincubation of the fluorophore with a biotinylated protein, can be visualized by FACS, as shown in FIG. 15. Binding to round 4 populations, unselected Ramos cells or IgM negative Ramos is not seen, indicating maturation of streptavidin binding.

Use of unaggregated streptavidin-FITC does not produce similar results, with the majority of round 6 cells not binding. This, in agreement with ELISA data, suggests that binding to streptavidin is due to avidity of the antibody binding to an array of antigen, rather than to a monovalent affinity. Higher affinity binders can be isolated by sorting for binding to non-aggregated streptavidin-FITC.

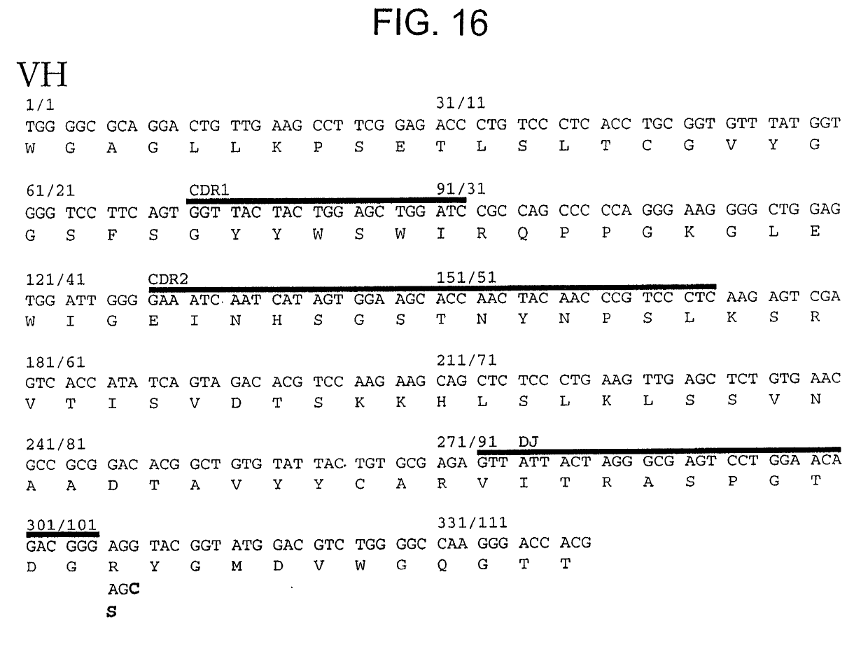

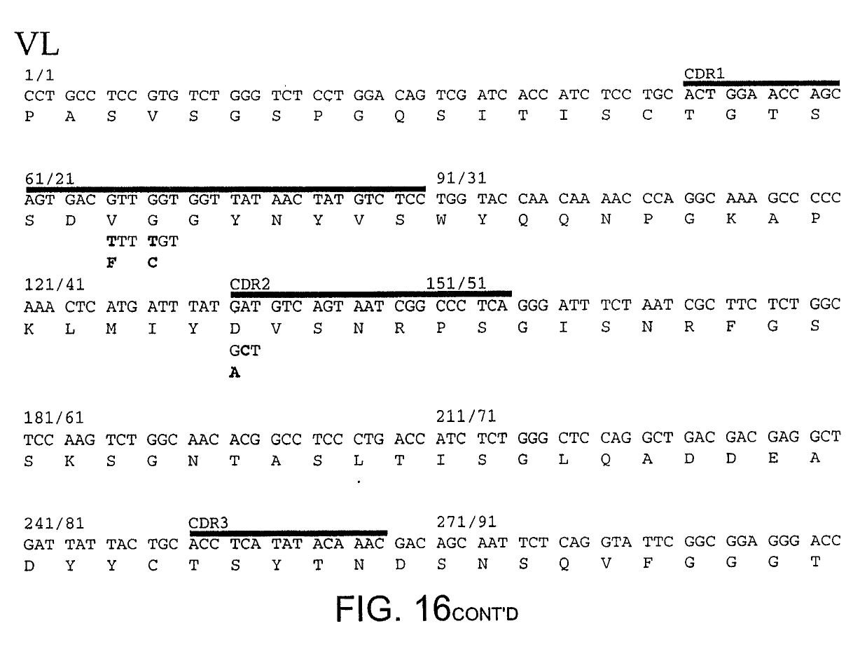

In order to determine the mutations responsible for the increased binding seen in round 6 cells over round 4 cells, the light and heavy chain antibody genes are amplified by PCR, and then sequenced. In comparison with round 4 cells, no changes in the heavy chain genes are seen, with the mutation R103S being conserved. In the light chain, mutations V23F and G24C are also conserved, but an additional mutation is present at position 46. Wild-type Ramos has an Aspartate at this position, while round 6 cells have an Alanine. Changes at this position are predicted to affect antigen binding, since residues in this region contribute to CDR2 of the light chain (FIG. 16). It seems likely that mutation D46A is responsible for the observed increase in binding to streptavidin seen in round 6 cells.

### Example 6

### In Vitro Maturation of Ramos Streptavidin Binders

Ram B→Ram C (Selecting with FITC-Poly-Streptavidin)

Approximately 5×10⁷ Ram B cells (derived from the Ramos cell line to bind Streptavidin coated microbeads) are washed with PBS and incubated on ice in 1 ml of PBS/BSA solution containing Poly-Streptavidin-FITC for 30 minutes (Poly-Streptavidin-FITC is made by adding streptavidin FITC (20 μg/ml protein content) to a biotinylated protein (10 μg/ml) and incubating on ice for a few minutes prior to the addition of cells).

The cells are then washed in ice cold PBS briefly, spun down and resuspended in 500 μl PBS.

The most fluorescent 1% of cells are sorted on a MoFlo cell sorter, and this population of cells is returned to tissue culture medium, expanded to approximately 5×10⁷ cells and the procedure repeated.

After four rounds of sorting with poly-Streptavidin-FITC the cells are binding weakly to Streptavidin-FITC. Sequence of the expressed immunoglobulin V regions from this Ramos cell population reveals that amino acid number 82a in framework three of the heavy chain V region had changed from Serine to Arginine. This population of cells is called Ram C.

Ram C→Ram D (Selecting with FITC-Streptavidin)

The next few rounds of cell sorting are done as described above but now using streptavidin-FITC (20 μg/ml protein content).

After three rounds of sorting using Streptavidin-FITC the sorted cell population (called Ram D) is binding more strongly to Streptavidin FITC as assayed by FACS. Sequence of the expressed V genes reveals a further amino acid change. In framework three the amino acid at position 65, originally a Serine, has changed to Arginine

Ram D→Ram E (Selecting with FITC-Streptavidin and Unlabelled Streptavidin Competition)

A subsequent sorting is done as described above using Streptavidin-FITC. However, after staining the cells on ice for 30 minutes, the cells are washed in ice cold PBS once and then resuspended in 0.5 mg/ml Streptavidin and incubated on ice for 20 minutes. This is in order to compete against the already bound Streptavidin-FITC, such that only Streptavidin-FITC that is strongly bound remains. The cells are then washed once in ice cold PBS and resuspended in 500 μl PBS prior to sorting the most fluorescent 1% population as before.

After repeating this sorting protocol a further two times the Ramos cell population (Ram E) appears to bind quite strongly to Streptavidin-FITC. These cells have acquired another amino acid change in framework one of the expressed heavy chain V gene; the amino acid at position 10 had changed from Glycine to Arginine.

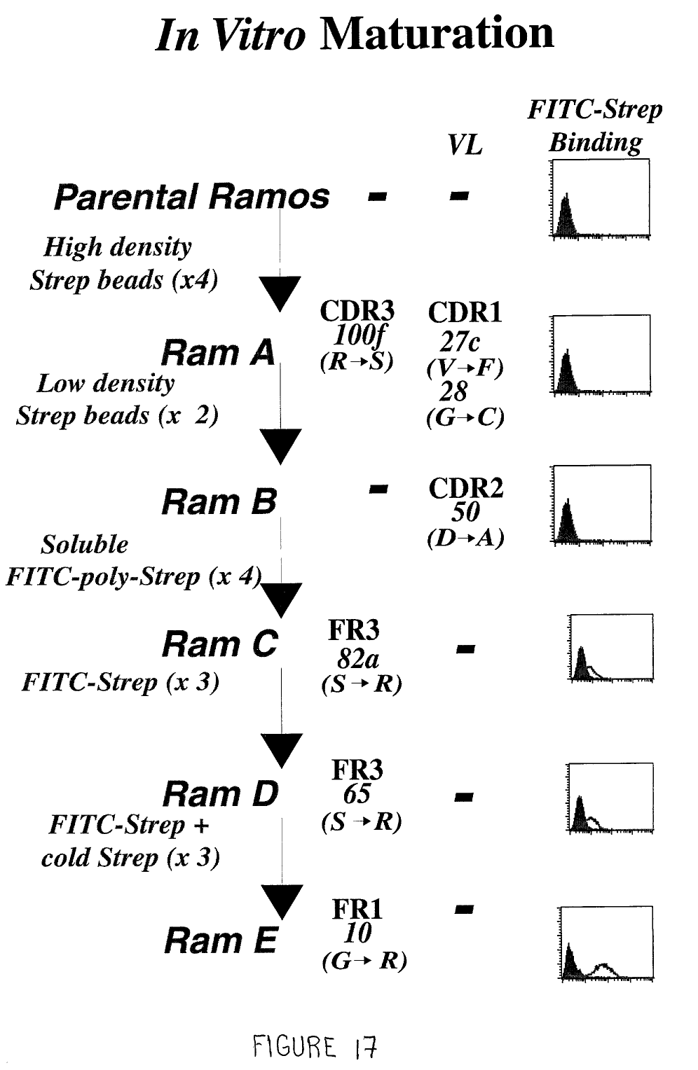

The results of the streptavidin maturation in Ramos cells are shown in FIG. 17.

ELISA Comparison

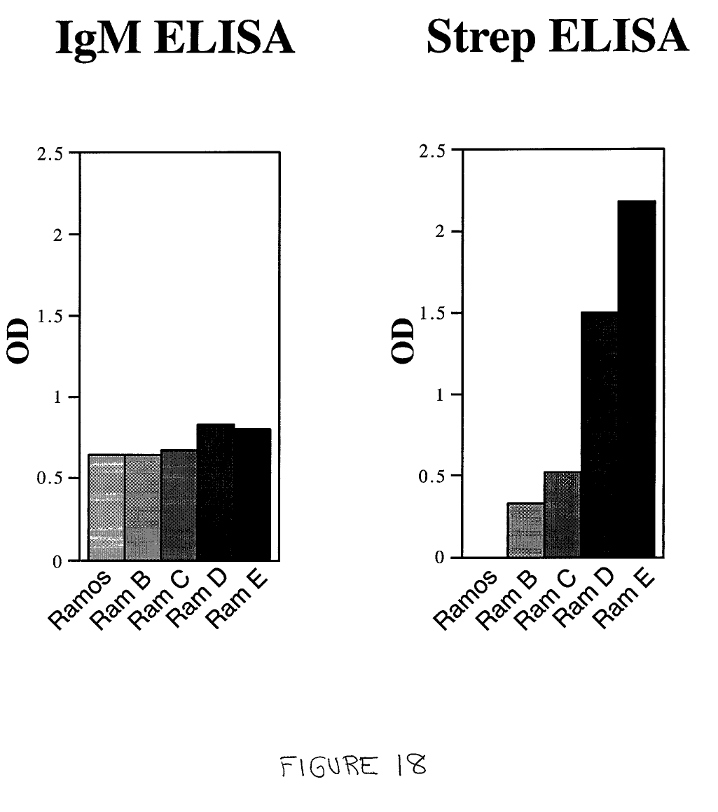

An ELISA assay performed with the supernatants of the various Ramos cell populations confirms that the IgM antibody expressed and secreted from Ramos cells has been matured in vitro to acquire a strong affinity for streptavidin. The results are set forth in FIG. 18.

### Example 7

### Construction of Transgene Comprising Hypermutation-Directing Sequences

It is known that certain elements of Ig gene loci are necessary for direction of hypermutation events in vivo. For example, the intron enhancer and matrix attachment region Ei/MAR has been demonstrated to play a critical role (Betz et al, 1994, Cell 77: 239-248). Moreover, the 3′ enhancer E3′ is known to be important (Goyenechea et al., 1997, EMBO J. 16: 3987-3994). However, these elements, while necessary, are not sufficient to direct hypermutation in a transgene.

In contrast, provision of Ei/MAR and E3′ together with additional Jκ-Cκintron DNA and Cκ is sufficient to confer hypermutability. A βG-Cκ transgene is assembled by joining an 0.96 Kb PCR-generated KpnI-SpeI β-globin fragment (that extends from −104 with respect to the β-globin transcription start site to +863 and has artificial KpnI and SpeI restriction sites at its ends) to a subfragment of LκΔ[3′Fl] (Betz et al., 1994, supra) that extends from nucleotide 2314 in the sequence of Max et al., 1981, J. Biol. Chem. 256: 5116-5120, through Ei/MAR, Cκ and E3′, and includes the 3′Fl deletion.

Hypermutation is assessed by sequencing segments of the transgene that are PCR amplified using Pfu polymerase. The amplified region extends from immediately upstream of the transcription start site to 300 nucleotides downstream of Jκ5.

This chimeric transgene is well targeted for mutation with nucleotide substitutions accumulating at a frequency similar to that found in a normal Igκ transgene. This transgene is the smallest so far described that efficiently recruits hypernutation and the results indicate that multiple sequences located somewhere in the region including and flanking Cκ (e.g., within 10 kb or less, preferably, within 9 kb or less) combine to recruit hypermutation to the 5′-end of the β-globin/Igκ chimaera.

The recruitment of hypermutation can therefore be solely directed by sequences lying towards the 3′-end of the hypermutation domain. However, the 5′-border of the mutation domain in normal Ig genes in the vicinity of the promoter, some 100-200 nucleotides downstream of the transcription start site. This positioning of the 5′-border of the mutation domain with respect to the start site remains even in the βG-Cκ transgene when the β-globin gene provides both the promoter and the bulk of the mutation domain. These results are consistent with findings made with other transgenes indicating that it is the position of the promoter itself that defines the 5′-border of the mutation domain.

The simplest explanation for the way in which some if not all the κ regulatory elements contribute towards mutation recruitment is to propose that they work by bringing a hypermutation priming factor onto the transcription initiation complex. By analogy with the classic studies on enhancers as transcription regulatory elements, the Igκ enhancers can work as regulators of hypermutation in a position- and orientation-independent manner. Indeed, the data obtained with the βG-Cκ transgene together with previous results in which E3′ was moved closer to Cκ (Betz et al., 1994, supra) reveal that the hypermutation-enhancing activity of E3′ is neither especially sensitive to its position or orientation with respect to the mutation domain.

Ei/MAR normally lies towards the 3′-end of the mutation domain. While deletion of Ei/MAR drastically reduces the efficacy of mutational targeting, its restoration to a position upstream of the promoter (and therefore outside the transcribed region) gives a partial rescue of mutation but without apparently affecting the position of the 5′-border of the mutational domain. Independent confirmation of these results was obtained in transgenic mice using a second transgene, tk-neo::Cκ in which a neo transcription unit (under control of the HSV tk promoter) is integrated into the Cκ exon by gene targeting in embryonic stem cells (Zou, et al., 1995, Eur. J. Immunol. 25: 2154-62). In this mouse, following Vκ-Jκ joining, the Igκ Ei/MAR is flanked on either side by transcription domains: the V gene upstream and tk::neo downstream. The tk-neo gene is PCR amplified from sorted germinal center B cells of mice homozygous for the neo insertion.

For the tk-neo insert in tk-neo::Cκ mice, the amplified region extends from residues 607 to 1417 [as numbered in plasmid pMCNeo (GenBank accession U43611)], and the nucleotide sequence determined from position 629 to 1329. The mutation frequency of endogenous VJκ rearrangements in tk-neo ::Cκ mice is determined using a strategy similar to that described in Meyer et al., 1996. Endogenous VJ_{κ5} rearrangements are amplified using a V_{κ} FR3 consensus forward primer (GGACTGCAGTCAGGTTCAGTGGCAGTGGG) and an oligonucleotide LκFOR (Gonzalez-Fernandez and Milstein, 1993, Proc. Natl. Acad. Sci. USA 90: 9862-9866) that primes back from downstream of the J_{κ} cluster.

Although the level of mutation of the tk-neo is low and it is certainly less efficiently targeted for mutation than the 3′-flanking region of rearranged V_{κ} genes in the same cell population, it appears that—as with normal V genes—the mutation domain in the neo gene insert starts somewhat over 100 nucleotides downstream of the transcription start site despite the fact that Ei/MAR is upstream of the promoter.

Thus, transgenes capable of directing hypermutation in a constitutively hypermutating cell line can be constructed using Ei/MAR, E3′ and regulatory elements as defined herein found downstream of Jκ. Moreover, transgenes can be constructed by replacement of, or insertion into, endogenous V genes, as in the case of the tk-neo ::Cκ mice, or by linkage of a desired coding sequence to the J_{κ} intron, as in the case of the βG-Cκ transgene.

### Example 8

### Selection of Constitutively Hypermutating Cell Line

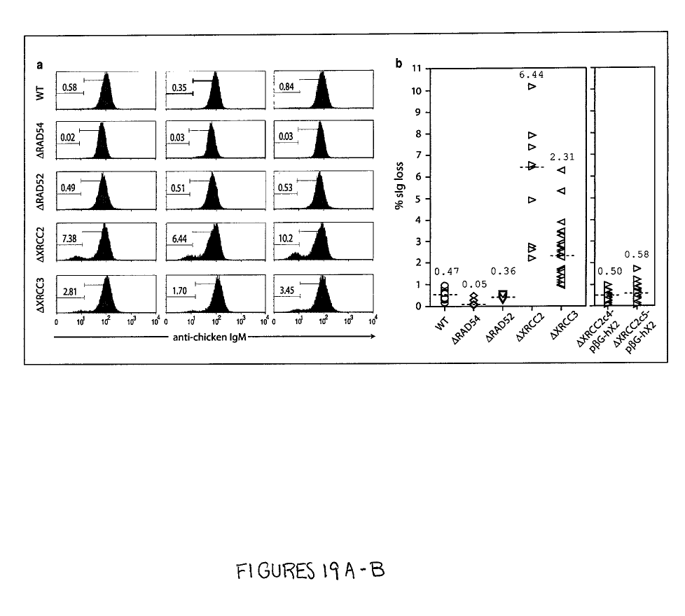

As described above, a small proportion of V gene conversion events can lead to the generation of a non-functional Ig gene, most frequently through the introduction of frameshift mutations. Thus, the generation of sIgM loss-variants in the chicken bursal lymphoma cell line, DT40, can be used to give an initial indication of IgV gene conversion activity. Compared to the parental DT40 line, a mutant that lacks Rad54 shows a considerably diminished proportion of sIgM-loss variants (FIG. 19). A fluctuation analysis performed on multiple clones reveals that the RAD54 line generates sIgM-loss variants at a frequency nearly tenfold less than that of parental DT40 while a RAD52 line generates sIgM-loss variants at a similar frequency to wild-type cells (FIG. 19). These observations are in keeping with earlier findings concerning gene conversion in RAD54 and RAD52-DT40 cells (Bezzubova et al., 1997; Cell 89: 185-193; Yamaguchi-Iwai et al., 1998, Mol Cell Biol 18: 6430-6435).

This analysis is extended to DT40 cells lacking Xrcc2 and Xrcc3. These Rad51 paralogues have been proposed to play a role in the recombination-dependent pathway of DNA damage repair (Liu et al., 1998, Mol Cell 1: 783-793); Johnson et al., 1999, Nature 401: 397-399; Brenneman et al., 2000, Mutat. Res. 459: 89-97; Takata et al., 2001, Mol Cell Biol. 21(8): 2858-66; Takata et al., 2000, Mol Cell Biol 20: 6476-6482). Rather than giving rise to a diminished abundance of sIgM-loss variants, the XRCC2 and XRCC3 lines show a much greater accumulation of loss variants than the parental line (FIG. 19). In the case of XRCC2-DT40, transfection of the human Xrcc2 cDNA under control of the human β-globin promoter causes the frequency of generation of sIgM-loss variants to revert to close to wild-type values. FIG. 19 shows the generation of sIgM-loss variants by wild-type and repair-deficient DT40 cells. Flow cytometric analyses of the heterogeneity of sIgM expression in cultures derived by 1 month of clonal expansion of single sIgM⁺ normal (WT) or repair-deficient (ΔRAD54, ΔRAD52, ΔXRCC2, ΔXRCC3) DT40 cells are shown in panel (a). An analysis of cultures derived from three representative sIgM⁺ precursor clones is shown for each type of repair-deficient DT40. The percentage of sIgM⁻ cells in each analysis is indicated with the fluorescence gate set as eight-fold below the center of the sIgM⁺ peak. Panel (b) shows fluctuation analysis of the frequency of generation of sIgM-loss variants. The abundance of sIgM-loss variants is determined in multiple parallel cultures derived from sIgM⁺ single cells after 1 month of clonal expansion; median percentages are noted above each data set and indicated by the dashed bar. The [pβG-hXRCC2]ΔXRCC2 transfectants analyzed are generated by transfection of pβG-hXRCC2 into sIgM⁺ DT40-ΔXRCC2 subclones that have 6.4% and 10.2% sIgM⁻ cells in the fluctuation analysis. The whole analysis is performed on multiple, independent sIgM⁺ clones (with distinct, though similar ancestral Vλ sequences) giving, for each repair-deficient line, average median frequencies at which sIgM-loss variants are generated after 1 month of WT (0.4%), ΔRAD54 (0.07%), ΔRAD52 (0.4%), ΔXRCC2 (6%) and ΔXCRCC3 (2%).

Since deficiency in both Xrcc2 and Xrcc3 is associated with chromosomal instability (see, e.g., Liu et al., 1998, supra; Cui et al., 1999, Mutat. Res. 434: 75-88; Deans et al., 2000, EMBO J 19: 6675-6685; Griffin et al., 2000, Nat Cell Biol 2: 757-761), it is possible that the increased frequency of sIgM-loss variants could reflect gross rearrangements or deletions within Ig loci. However, Southern blot analysis of 24 sIgM⁻ subclones of XRCC3-DT40 does not reveal any loss or alteration of the 6 kb SalI-BamHI fragment containing the rearranged V_{λ}.

Therefore, to ascertain whether more localized mutations in the V gene could account for the loss of sIgM expression, the rearranged V_{λ} segments in populations of sIgM cells that are sorted from wild-type, ΔXRCC2- and ΔXRCC3-DT40 subclones after one month of expansion are cloned and sequenced.

Cell Culture, Transfection and Analysis

DT40 subclone CL18 and mutants thereof are propagated in RPMI 1640 supplemented with 7% fetal calf serum, 3% chicken serum (Life Technologies), 50 μM 2-mercaptoethanol, penicillin and streptomycin at 37 C. in 10% CO₂. Cell density was maintained at between 0.2-1.0×10⁶ ml⁻¹ by splitting the cultures daily. The generation of the DT40 derivatives carrying targeted gene disruptions has been described elsewhere (Bezzubova et al., 1997, supra; Yamnaguchi-Iwai et al., 1998, supra; Takata et al., 2001, supra; Takata et al., 2000, supra; Takata et al., 1998, EMBO J 17: 5497-5508). Transfectants of ΔXRCC2-DT40 harboring a pSV2-neo based plasmid that contains the XRCC2 open reading frame (cloned from HeLa cDNA) under control of the β-globin promoter are generated by electroporation.

CL18 is an sIgM⁻ subclone of DT40 and is the parental clone for the DNA repair-mutants described here. Multiple sIgM⁺ subclones are obtained from both wild-type and repair-deficient mutants using a Mo-Flo (Cytomation) sorter after staining with FITC-conjugated goat anti-chicken IgM (Bethyl Laboratories). There is little variation in the initial Vλ sequence expressed by all the sIgM⁺ DT40-CL18 derived repair-deficient cells used in this work since nearly all the sIgM⁺ derivatives have reverted the original CL18 Vλ frameshift by gene conversion using the ΨV8 donor (which is most closely related to the frameshifted CL18 CDR1).

Mutation Analysis

Genomic DNA is PCR amplified from 5000 cell equivalents using Pfu Turbo (Stratagene) polymerase and hotstart touchdown PCR [8 cycles at 95 C. 1′; 68-60 C. (at 1 C. per cycle) 1 min.; 72 C. 1 min., 30 sec.; 22 cycles @94 C., 30 sec.″; 60 C., 1 min.; 72 C. 1 min., 30 sec.]. The rearranged Vλ is amplified using CVLF6 (5′-CAGGAGCTCGCGGGGCCGTCACTGATTGCCG; priming in the leader-Vλ intron) and CVLR3 (5′-GCGCAAGCTTCCCCAGCCTGCCGCCAAGTCCAAG; priming back from 3′ of Jλ); the unrearranged Vλ1 using CVLF6 with CVLURR1 (5′GGAATTCTCAGTGGGAGCAGGAGCAG); the rearranged V_{H} gene using CVH1F1 (5′-CGGGAGCTCCGTCAGCGCTCTCTGTCC) with CJH1R1 (5′-GGGGTACCCGGAGGAGACGATGACTTCGG) and the C_{λ} region using CJC1R1F (5′-GCAGTTCAAGAATTCCTCGCTGG; priming from within the J_{λ}-C_{λ} intron) with CCMUCLAR (5′-GGAGCCATCGATCACCCAATCCAC; priming back from within C_{λ}). After purification on QI Aquick spin columns (Qiagen), PCR products are cut with the appropriate restriction enzymes, cloned into pBluescriptSK and sequenced using the T3 or T7 primers and an ABI377 sequencer (Applied Biosystems). Sequence alignment (Bonfield et al., 1995, supra) with GAP4 allowed identification of changes from the consensus sequence of each clone.

All sequence changes are assigned to one of three categories: gene conversion, point mutation or an ambiguous category. This discrimination rests on the published sequences of the V_{λ} pseudogenes that could act as donors for gene conversion. The database of such donor sequences is taken from Reynaud et al., 1987, Cell 48: 379-388, but implementing the modifications (McCormack et al., 1993, Mol Cell Biol. 13: 821-830) pertaining to the Igλ G4 allele appropriate (Kim et al. 1990, Mol Cell Biol 10: 3224-3231) to the expressed Igλ in DT40. (The sequences/gene conversions identified in this work supported the validity of this ΨVλ sequence database). For each mutation the database of Vλ pseudogenes is searched for potential donors. If no pseudogene donor containing a string 9 bp can be found then it is categorized as an untemplated point mutation. If a such a string is identified and there are further mutations which can be explained by the same donor, then all these mutations are assigned to a single gene conversion event. If there are no further mutations then the isolated mutation could have arisen through a conversion mechanism or could have been untemplated and is therefore categorized as ambiguous.

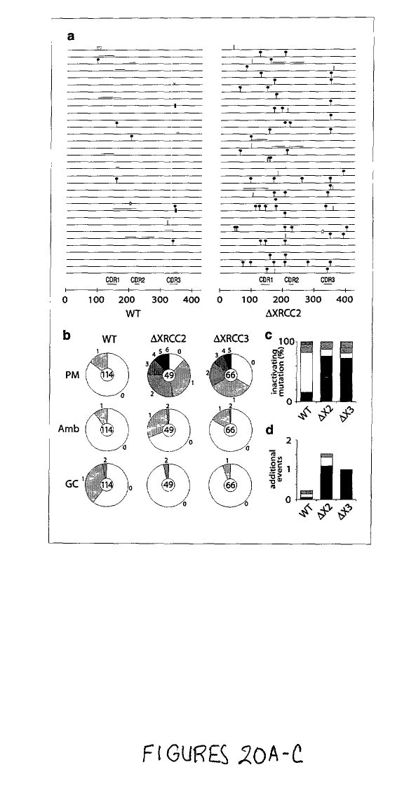

With regard to the V_{λ} sequences cloned from the sIgM⁻ subpopulations sorted from multiple wild-type DT40 clones, 67% carry mutations: in the majority (73%) of cases, these mutations render the V_{λ} obviously non-functional, as shown in FIG. 20. Presumably, most of the remaining sIgM cells carry inactivating mutations either in V_{H} or outside the sequenced region of Vλ. FIG. 20 shows analyses of V_{λ} sequences cloned from sIgM-loss variants. In panel (a), comparison of V_{λ} sequences obtained from sIgM-loss cells that have been sorted from parental sIgM⁺ clones of normal or Xrcc2-deficient DT40 cells after 1 month of clonal expansion. Each horizontal line represents the rearranged V_{λ} [symbol] λ (427 bp) with mutations classified as described above as point mutations (lollipop), gene conversion tracts (horizontal bar above line) or single nucleotide substitutions which could be a result of point mutation or gene conversion (ambiguous, vertical bar). Hollow boxes straddling the line depict deletions, triangles indicate a duplications. Pie charts are shown in panel (b), depicting the proportion of Vλ sequences that carry different numbers of point mutations (PM), gene conversions (GC) or mutations of ambiguous origin (Amb) amongst sorted sIgM-loss populations derived from wild-type, ΔXRCC2 or ΔXRCC3 DT40 sIgM⁺ clones after 1 month of clonal expansion. The sizes of the segments are proportional to the number of sequences carrying the number of mutations indicated around the periphery of the pie. The total number of Vλ sequences analyzed is indicated in the center of each pie with the data compiled from analysis of four subclones of wild-type DT40, two of ΔXRCC2-DT40 and three of ΔXRCC3-DT40. Deletions, duplications and insertions are excluded from this analysis; in wild-type cells, there are additionally 6 deletions, 1 duplication and 1 insertion. There are no other events in ΔXRCC2-DT40 and a single example each of a 1 bp deletion and a 1 bp insertion in the ΔXRCC3-DT40 database.

Causes of Vλ gene inactivation in wild-type, ΔXRCC2 (ΔX2) and ΔXRCC3 (ΔX3) DT40 cells expressed as a percentage of the total sequences that contained an identified inactivating mutation are set forth in panel (c): Missense mutation (black). Gene conversion-associated frameshift (white). Deletions, insertions or duplication-associated frameshift (grey). Additional mutational events associated with each inactivating mutation are then shown in (d). The data are expressed as the mean number of additional mutations associated with each inactivating mutation with the type of additional mutation indicated as in panel (c). Thus, ΔXRCC2-DT40 has a mean of 1.2 additional point mutations in addition to the index inactivating mutation whereas wild-type DT40 has only 0.07.

As detailed above, the mutations can be classified as being attributable to gene conversion templated by an upstream Vλ pseudogene, to non-templated point mutations or as falling into an ambiguous category. Most (67%) of the inactivating mutations are due to gene conversion although some (15%) are stop codons generated by non-templated point mutations demonstrating that the low frequency of point mutations seen here and elsewhere (Buerstedde et al., 1985, EMBO J. 9: 921-927; Kim et al., 1990, supra) in DT40 cells is not a PCR artifact but rather reveals that a low frequency of point mutation does indeed accompany gene conversion in wild-type DT40 .

A strikingly different pattern of mutation is seen in the Vλ sequences of the sIgM-loss variants from ΔXRCC2-DT40. Nearly all the sequences carry point mutations, typically with multiple point mutations per sequence. A substantial shift towards point mutations is also seen in the sequences from the sIgM⁻ ΔXRCC3-DT40 cells. Thus, whereas a Vλ-inactivating mutation in wild-type DT40 is most likely to reflect an out of frame gene conversion tract, in ΔXRCC2/3 it is likely to be a missense mutation (FIG. 20c). Furthermore, whereas most of the nonfunctional Vλ sequences obtained from sorted sIgM-loss variants of ΔXRCC2-DT40 (53%) or ΔXRCC3-DT40 (64%) carry additional point mutations in addition to the Vλ-inactivating mutation, such hitchhiking is only rarely observed in the nonfunctional Vλ sequences from the parental DT40 line (7%; FIG. 20d).

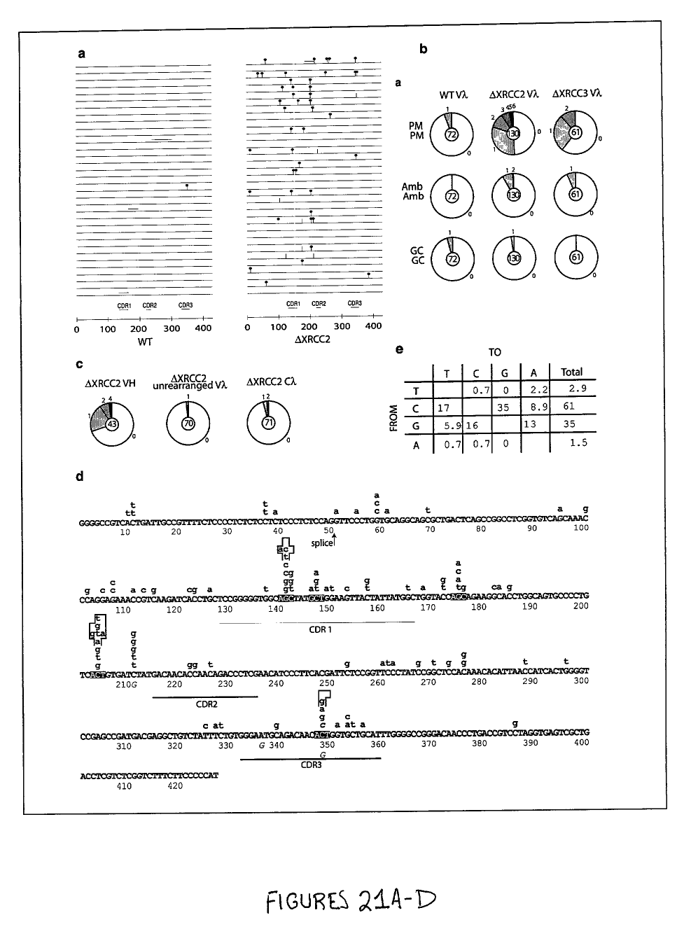

All these observations suggest that the high prevalence of sIgM-loss variants in ΔXRCC2/3-DT40 cells simply reflects a very high frequency of spontaneous IgV gene hypermutation in these cells. FIG. 21 represents analyses of Ig sequences cloned from unsorted DT40 populations after one month of clonal expansion. The V_{λ} sequences obtained from representative, wild-type and ΔXRCC2 DT40 clones are presented in panel (a) with symbols as in FIG. 20. In panel (b), pie charts are shown depicting the proportion of the V_{λ} sequences carrying different numbers of the various types of mutation as indicated. The data are pooled from analysis of independent clones: wild-type (two clones), ΔXRCC2 (four clones) and ΔXRCC3 (two clones). In addition to the mutations shown, one ΔXRCC2-DT40 sequence contained a 2 bp insertion in the leader intron which was not obviously templated from a donor pseudogene and one ΔXRCC3-DT40 sequence carried a single base pair deletion also in the leader intron.

Mutations at other loci of ΔXRCC2-DT40 are shown in panel (c). Pie charts depict the proportion of sequences derived from 1 month-expanded ΔXRCC2-DT40 cells that carry mutations in the rearranged V_{H} (272 bp extending from CDR1 to the end of J_{H}) of the rearranged heavy chain of, in the unrearranged Vλ1 on the excluded allele (458 bp) and in the vicinity of Cλ (425 bp extending from the Jλ-Cλ intron into the first 132 bp of Cλ). Analysis of known V_{H} pseudogene sequences (Reynaud et al., 1989, Cell 59: 171-183) does not indicate that any of the mutations observed in the rearranged V_{H} are due to gene conversion, strongly suggesting that they are due to point mutation although this assignment cannot be regarded as wholly definitive. The mutation prevalences in these data sets are: 1.6×10⁻³ mutations bp⁻¹ for V_{H},0.03×10⁻³ for the unrearranged Vλ1 and 0.13×10⁻³ for Cλ as compared to 2.0×10⁻³ for point mutations in the rearranged Vλ1 in ΔXRCC2-DT40, 0.13×10⁻³ for point mutations in rearranged Vλ1 in wild-type DT40 and 0.04×10⁻³ for background PCR error.

The distribution of point mutations across Vλ1 is shown in panel (d). The ΔXRCC2-DT40 consensus is indicated in upper case with the first base corresponding to the 76^{th} base pair of the leader intron. Variations found in the ΔXRCC3-DT40 consensus are indicated in italic capitals below. The mutations are shown in lower case letters above the consensus with those from ΔXRCC2-DT40 in black and those from ΔXRCC3-DT40 in mid-grey. All mutations falling into the point mutation and ambiguous categories are included. Correction has been made for clonal expansion as described previously (Takata et al, 1998) so each lower case letter represents an independent mutational event. The majority of the 27 mutations thereby removed from the original database of 158 are at one of the seven major hotspots; the correction for clonality will, if it gives rise to any distortion, lead to a underestimate of hotspot dominance. Of the seven major hotspots (identified by an accumulation of 5 mutations), five conform to the AGY consensus sequence on one of the two strands as indicated with black boxes. Nucleotide substitution preferences (given as a percentage of the database of 131 independent events) as shown in panel (e) are deduced from the point mutations in sequences from unselected ΔXRCC2- and ΔXRCC3-DT40. A similar pattern of preferences is evident if the ΔXRCC2/ΔXRCC3 databases are analyzed individually.

The spontaneous Vx mutation frequency in wild-type and ΔXRCC2/3-DT40 cells is analyzed by PCR amplifying the rearranged V₈₀ segments from total (unsorted) DT40 populations that have been expanded for 1 month following subcloning. The result reveals that there is indeed a much higher spontaneous accumulation of mutations in the ΔXRCC2 and ΔXRCC3 cells than in the parental DT40 (FIG. 21a, b). In ΔXRCC2-DT40 cells, mutations accumulate in V_{λ} at a rate of about 0.4×10⁻⁴ bp⁻¹.generation⁻¹ (given an approximately 12 hour division time), a value similar to that seen in the constitutively mutating human Burkitt lymphoma line Ramos.

Somatic hypermutation in germinal center B cells in man and mouse is preferentially targeted to the rearranged immunoglobulin V_{H} and V_{L} segments. A similar situation applies to the point mutations in ΔXRCC2-DT40 cells. Thus, a significant level of apparent point mutation is also seen in the productively rearranged V_{H}1 gene (FIG. 3c). However, this does not reflect a general mutator phenotype since mutation accumulation is much lower in Cλ than in the rearranged V_{λ} and is also low in the unrearranged V_{λ} on the excluded allele where the apparent mutation rate does not rise above the background level ascribable to the PCR amplification itself (FIG. 21c).

The distribution of the mutations over the V_{λ} domain in ΔXRCC2-DT40 cells is strikingly non-random. The mutations, which are predominantly single nucleotide substitutions, show preferential accumulation at hotspots that conform to an AGY (Y=pyrimidine) consensus on one of the two DNA strands (FIG. 21d). They also occur overwhelmingly (96%) at G/C. This G/C-biased, hotspot-focused hypermutation in ΔXRCC2-DT40 cells, although exhibiting somewhat less of a bias in favor of nucleotide transitions, is strikingly similar to the pattern of V gene hypermutation described in cultured human Burkitt lymphoma cells as well as that occurring in vivo in frog, shark and Msh2-deficient mice (Rada et al., 1998, Immunity 9: 135-141; Diaz et al, 2001, Philos. Trans. R. Soc. Lond. B. Biol. Sci. 356: 67-72). The IgV gene hypermutation that occurs in vivo in man and normal mice appears, as previously discussed, to be achieved by this hotspot-focused G/C biased component acting in concert with a mechanism that targets A/T (FIG. 21e).

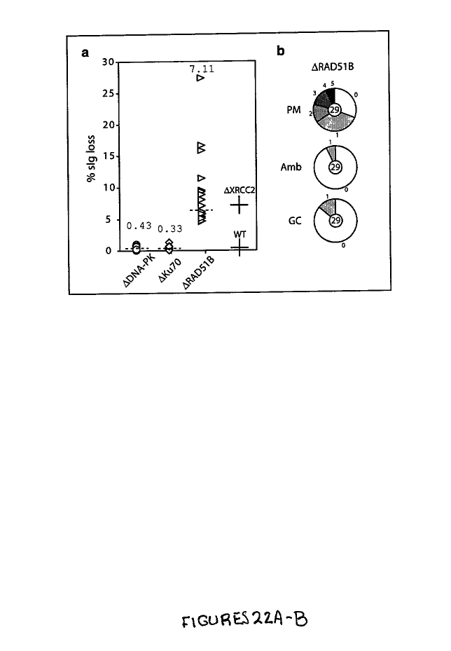

Thus, whereas the DT40 chicken bursal lymphoma line normally exhibits a low frequency of IgV diversification by gene conversion, a high frequency of constitutive IgV gene somatic mutation (similar in nature to that occurring in human B cell lymphoma models) can be elicited by ablating Xrcc2 or Xrcc3. This provides strong support to the earlier proposal that IgV gene conversion and hypermutation might constitute different ways of resolving a common DNA lesion (Maizels et al., 1995, Cell 83: 9-12; Weill et al., 1996, Immunol. Today 17: 92-97). Recent data suggest that the initiating lesion could well be a double strand break (Sale and Neuberger, 1998, Immunity 2: 859-869; Papavasilou and Schatz, 2000, Nature 408: 216-221; Bross et al., 2000, Immunity 13: 589-597) it would therefore appear significant that both Xrcc2 and Xrcc3 have been implicated in a recombination-dependent pathway of DNA break repair (Liu et al., 1998, supra; Johnson et al., 1999, supra; Pierce et al., 1999, Genes Dev 13: 2633-2638; Brennerman et al., 2000, supra; Takata et al., 2001, supra). Indeed, a similar induction of IgV gene hypermutation in DT40 cells is achieved by ablating another gene (RAD51B) whose product is implicated in recombination-dependent repair of breaks (Takata et al, 2000, supra) but not by ablating genes for Ku70 and DNA-PK_{cs} which are involved in non-homologous end-joining. FIG. 22 shows the analysis of sIgM-loss variants in DT40 cells deficient in DNA-PK, Ku70 and Rad51B. Fluctuation analysis of the frequency of generation of sIgM-loss variants after 1 month of clonal expansion is shown in panel (a). The median values obtained with wild-type and ΔXRCC2 DT40 are included for comparison. Pie charts depicting the proportion of V_{λ} sequences amplified from the sIgM-loss variants derived from two sIgM⁺ Rad51B-deficient DT40 clones that carry various types of mutation as indicated are shown in panel (b). In addition, one sequence carried a 9 bp deletion, one carried a 4 bp duplication and one carried a single base pair insertion.

The results, however, do not simply suggest that, in the absence of Xrcc2, a lesion which would normally be resolved by gene conversion is instead resolved by a process leading to somatic hypermutation. First, ΔXRCC2-DT40 cells retain the ability to perform IgV gene conversion, albeit at a somewhat reduced level (FIG. 21b). Second, the frequency of hypermutation in ΔXRCC2-DT40 cells is about an order of magnitude greater than the frequency of gene conversion in the parental DT40 line. It is therefore likely that, in normal DT40 cells, only a minor proportion of the lesions in the IgV gene are subjected to templated repair from an upstream pseudogene thereby leading to the gene conversion events observed. We believe that the major proportion of the lesions are subjected to a recombinational repair using the identical V gene located on the sister chromatid as template and which is therefore ‘invisible’. This would be consistent with the observations of Papavasiliou and Schatz, 2000, Nature 408: 216-221, who found that detectable IgV gene breaks in hypermutating mammalian B cells are restricted to the G2/S phase. In the absence of Xrcc2, Xrcc3 or Rad51B, we propose that the ‘invisible’ sister chromatid-dependent recombinational repair is perverted, resulting in hypermutation. Whether this hypermutation reflects that the sister chromatid-dependent recombinational repair becomes error-prone in the absence of Xrcc2/3 or whether it reflects an inhibition of such repair thereby revealing an alternate, non-templated mechanism of break resolution is an issue that needs to be addressed. This question is not only important for an understanding of the mechanism of hypermutation but can also provide insight into the physiological function of the Rad51 paralogues.

### Example 9

### Isolation of Naturally-occurring Constitutively Hypermutating EBV Positive BL Cell Lines

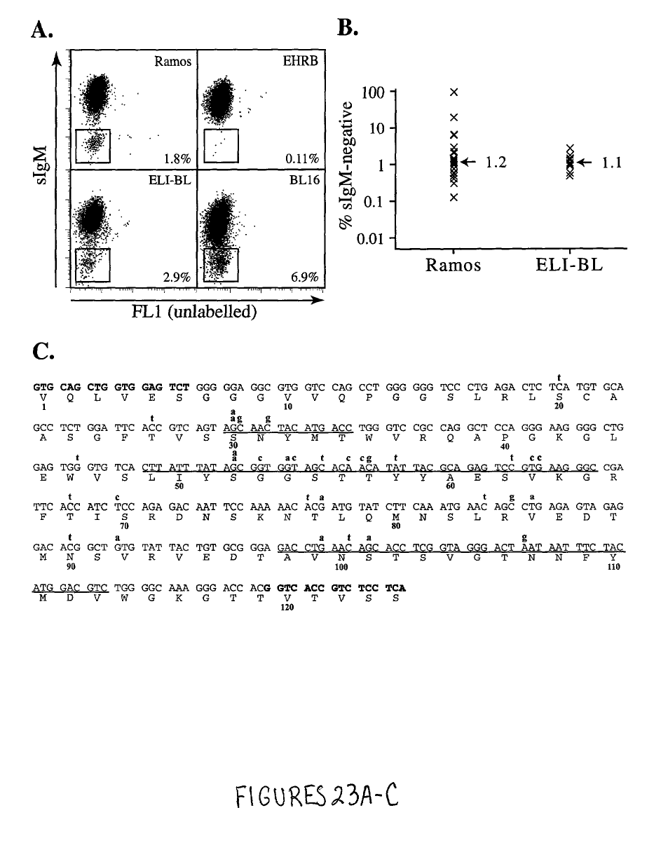

A survey of naturally occurring EBV⁺ BL cell lines revealed an absence of a clearly identifiable population of sIgM-loss variants amongst many of them (e.g. Akata, BL74, Chep, Daudi, Raji, and Wan). However, a clear sIgM^{-/low} population was noted in two of these EBV⁺ cell lines, ELI-BL and BL16, suggesting an intrinsic hypermutation capacity. sIgM expression profiles of Ramos, EHRB, ELI-BL, and BL16 are shown in FIG. 23a. The sIgM^{-/low} cell population is boxed and the percentage of cells therein indicated. Each dot represents one cell. Note that the sizable sIgM^{-/low} population in BL16 is in part due to less intensely staining positive cells, which also occluded fluctuation analyses. ELI-BL harbors a type 2 EBV, resembles germinal center B cells, and expresses a latency gene repertoire consisting only of EBNA1 and the non-coding EBER and Bam A RNAs (Rowe, et al., 1987, EMBO J. 6: 2743-51) BL16 also contains a type 2 virus but, in contrast to ELI-BL, it appears more LCL-like and expresses a full latency gene repertoire (Rooney et al., 1984, Int J Cancer 34: 339-48; Rowe et al., 1987, supra).

Although a clear sIgM^{-/low} population was visible in ELI-BL and BL16 cultures, it was important to address whether these variants could be attributed to bonafide hypermutation. This was assessed by fluctuation analysis. In brief, subclones were transferred to 24 or 48 well plates, maintained with fresh medium for 3 to 8 weeks, and analyzed by washing cells (1−2.5×10⁵) twice in PBS/3% FBS, staining (30 min on ice) with the relevant antibody or antibody combination (below) and again washing prior to analysis of at least 10⁴ cells by flow cytometry (FACSCalibur, Becton Dickinson). Antibodies used were R-phycoerythrin-conjugated, goat anti-human IgM (μ-chain specific; Sigma), fluorescein isothiocyanate (FITC)-conjugated, mouse monoclonal anti-Ramos idiotype [ZL16/1 (Zhang et al., 1995, Ther. Immunol. 2: 191-202); provided generously by M. Cragg and M. J. Glennie, Tenovus Research Laboratory, Southampton], and FITC-conjugated, goat anti-mouse IgM (Southern Biotechnology Associates, Inc.). Data were acquired and analyzed using CellQuest software (Becton Dickinson).

Unless noted otherwise, cells compared in fluctuation analyses were derived, cultured, and analyzed in parallel. The median (as opposed to the mean) percentage of sIgM-loss variants amongst a number of identically-derived (sub)clones is used as an indicator of a cells somatic hypermutation capacity to minimize the effects of early mutational events Fluctuation analysis of ELI-BL subclones revealed that the sIgM^{-/low} variants were indeed being generated at high frequency during in vitro culture (FIG. 23b; each cross represents the percentage of cells falling within the sIgM^{-/low} window following a 1 month outgrowth of a single subclone; the median percentages are indicated), and V_{H} sequence analysis, in the case of BL16 subclones, confirmed that this instability reflected somatic hypermutation (FIG. 23c). Base substitution mutations are indicated in lower case letters above the 338 bp consensus DNA sequence in triplets of capital letters. Complementarity-determining regions and partial PCR primer sequences are underlined and emboldened, respectively. The corresponding amino acid sequence is indicated by single capital letters. This consensus sequence differs at two positions from GenBank entry gi.2253343 [TCA (Ser20)-TCT and AGC (Ser55)-ACC (Thr)].

Considerable V_{H} sequence diversity, including several sequences with multiple base substitution mutations, and an overall high V_{H} mutation frequency indicated that hypermutation is ongoing in BL16. Moreover, despite the relatively small number of V_{H} sequences sampled, one dynastic relationship could be inferred [1^{st} mutation at Gly54 (GGT-GAT); 2^{nd} mutation at Val92 (GTG-ATG)]. Finally, like Ramos, most of the BL16 V_{H} base substitution mutations occurred at G or C nucleotides (24/33 or 73%) and clustered within the complementarity determining regions (underlined in FIG. 23c). Thus, several hallmarks of ongoing hypermutation were also distinguishable in two natural EBV⁺ BL cell lines, one expressing a limited latency gene repertoire and the other expressing a full combination. It was therefore clear that somatic hypermutation can proceed unabated even in the presence of EBV.

All references cited herein are incorporated by reference herein.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

What is claimed is:

1. A method for obtaining a cell which directs constitutive hypermutation of a target nucleic acid sequence within the cell, comprising screening a cell population for ongoing target sequence diversification and selecting a cell in the cell population in which the rate of mutation of the target sequence exceeds the rate of mutations in non-target sequences in the cell by a factor of 100 or more.

2. The method according to claim 1, wherein the cell population comprises lymphoid cells.

3. The method according to claim 2, wherein the cell population comprises is immunoglobulin-expressing cells.

4. The method according to claim 1, wherein the cell expresses a gene product encoded by the target nucleic acid sequence; and wherein screening comprises selecting for a change in the expression of the gene product.

5. The method according to claim 1, wherein the change is a loss of expression of the gene product.

6. The method according to claim 4, wherein the cell expresses the gene product on its surface.

7. The method according to claim 1, wherein the cell is from a cell line generated from a cell which hypermutates in vivo.

8. A method according to claim 7, wherein the cell is from a cell line selected from the group consisting of a Burkitt lymphoma cell line, a follicular lymphoma cell line, and a diffuse large cell lymphoma cell line.

9. The method according to claim 1, further comprising the steps of isolating one or more of selected cells and comparing the rate of accumulation of mutations in the target sequence in the one or more cells with the rate of mutation of non-target sequences.

10. The method according to claim 1, wherein the target sequence is an immunoglobulin V-gene sequence.

11. The method according to claim 5, wherein the gene product is an immunoglobulin.

12. The method according to claim 1, wherein mutation rates are determined by sequencing the target sequence in each of a plurality of cells from the cell population

13. The method according to claim 5, wherein the cells are contacted with an antibody which specifically binds to the gene product to identify one or more cells which do not bind to the antibody.

14. The method according to claim 1, wherein the cells are exposed to a mutagen.

15. The method according to claim 1, wherein the cells express a sequence-modifying gene product.

16. The method according to claim 1, wherein the cells comprise one or more mutated sequences providing the cells with a higher rate of mutation than cells without the one or more mutated sequences.

17. The method according to claim 16, wherein the rate of mutation is at least two-fold higher in the cells comprising the one or more mutated sequences than in the cells without the one or more mutated sequences.

18. The method according to claim 17, wherein the rate of mutation is at least ten-fold higher.

19. The method according to claim 16, wherein the one or more mutated sequences are genetically engineered into the cells.

20. The method according to claim 16, wherein the one or more mutated sequences comprises one or more mutated DNA repair genes.

21. The method according to claim 16, wherein the cells comprising the one or more mutated sequences express at least 10% less of one or more DNA repair proteins than cells without the one or more mutated sequences.

22. The method according to claim 20, wherein the one or more DNA repair genes are selected from the group consisting of Rad51, Rad 51 analogues, Rad51 paralogues, and combinations thereof.

23. The method according to claim 20, wherein the DNA repair genes are selected from the group consisting of Rad51b, Rad51c, and analogues, paralogues, and combinations thereof.

24. A method for preparing a mutated form of a gene product, comprising the steps of:
a) expressing a nucleic acid encoding the gene product, the nucleic acid operably linked to a hypermutation control sequence, in a population of constitutively hypermutating cells in which the rate of mutation of nucleic acids linked to the control sequence exceeds the rate of mutations in sequences not linked to the control sequence by a factor of 100 or more; and
b) identifying a cell or cells within the population of cells which express a mutated form of the gene product.

25. The method according to claim 24, further comprising
c) establishing one or more clonal populations of cells from the cell or cells identified in step (b), and selecting from the clonal populations a cell or cells which expresses the mutated form of the gene product.

26. The method according to claim 24, wherein the cell or cells constitutively hypermutate an endogenous V gene locus.

27. The method according to claim 24, wherein the mutated form of the gene product binds to a biomolecule to which the non-mutated form of the gene product does not bind.

28. The method according to claim 24, wherein the mutated form of the gene product is unable to bind to a biomolecule under conditions in which the non-mutated form of the gene product binds to the biomolecule.

29. The method according to claim 24, wherein the mutated form of the gene product comprises an at least two-fold greater ability to bind to a biomolecule to which the non-mutated form of the gene product binds.

30. The method according to claim 24, wherein the mutated form of the gene product comprises an at least two-fold lower ability to bind to a biomolecule to which the non-mutated form of the gene product binds.

31. The method according to claim 24, wherein the gene product is an enzyme.

32. The method according to claim 24, wherein the gene product performs a catalytic activity in the presence of a substrate and wherein the catalytic activity of the mutated gene product is increased at least two-fold compared to the catalytic activity of the non-mutated gene product.

33. The method according to claim 24, wherein the gene product performs a catalytic activity in the presence of a substrate and wherein the catalytic activity of the mutated gene product is decreased at least two-fold compared to the catalytic activity of the non-mutated gene product.

34. The method according to claim 24, wherein the hypermutation control sequence comprises a sequence occurring 3′ of a J gene cluster, said sequence comprising the Jκ-Cκ intron sequence, Cκ, and the E3′ enhancer element, and wherein said Jκ-Cκ intron sequence comprises the Ei/MAR enhancer element sequence.

35. The method according to claim 34, wherein the sequence 3′ of Cκ and 5′ of E3′ comprises a 7.34 kb deletion.

36. The method according to claim 24, wherein the nucleic acid encoding the gene product is an exogenous sequence operably linked to an endogenous control sequence.

37. The method according to claim 36, wherein the exogenous gene is operably linked to the Jκ intron.

38. The method according to claim 36, wherein the exogenous sequence is a heterologous coding sequence not naturally found in the cell or cells.

39. The method according to claim 36, wherein an endogenous V region coding sequence is replaced by a heterologous coding sequence not naturally found in the cell or cells' genomes.

40. The method according to claim 24, wherein the gene product is an immunoglobulin.

41. The method according to claim 24, wherein the gene product is a DNA binding protein.

42. A cell for directing constitutive hypermutation of a target gene, wherein the cell is a genetically manipulated chicken bursal lymphoma cell in which the rate of nucleic acid mutation at a target sequence in the cell operably linked to a hypermutation control sequence for directing mutations to the target sequence exceeds the rate of mutations in non-target nucleic acids by a factor of 100 or more.

43. The cell according to claim 42, wherein the chicken bursal lymphoma cell is a DT40 cell.

44. A cell selected from the group consisting of Δ xrcc2 DT40 and Δxrcc3 DT40.
